SEQUENCE LISTING (10 sequences)

SEQ ID NO: 1 - protein, 216 aa, synthetic construct
  REGION 1..216: note = antibody light chain
QSALTQPASVSGSPGQSITISCTGTGSDVGSYNLVSWYQQHPGKAPKLMIYGDSERPSGVSNRFSGSKSGNTASLTISGLQAEDEADYYCSSYAGSGIYVFGTGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 2 - protein, 445 aa, synthetic construct
  REGION 1..445: note = antibody heavy chain
EVQLLESGGGLVQPGGSLRLSCAASGFTFSTYAMGWVRQAPGKGLEWVSSIGASGSQTRYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 14 aa, synthetic construct
  REGION 1..14: note = complementarity-determining region
TGTGSDVGSYNLVS

SEQ ID NO: 4 - protein, 7 aa, synthetic construct
  REGION 1..7: note = complementarity-determining region
GDSERPS

SEQ ID NO: 5 - protein, 10 aa, synthetic construct
  REGION 1..10: note = complementarity-determining region
SSYAGSGIYV

SEQ ID NO: 6 - protein, 5 aa, synthetic construct
  REGION 1..5: note = complementarity-determining region
TYAMG

SEQ ID NO: 7 - protein, 14 aa, synthetic construct
  REGION 1..14: note = complementarity-determining region
SIGASGSQTRYADS

SEQ ID NO: 8 - protein, 7 aa, synthetic construct
  REGION 1..7: note = complementarity-determining region
LAIGDSY

SEQ ID NO: 9 - protein, 110 aa, synthetic construct
  REGION 1..110: note = antibody light chain variable region
QSALTQPASVSGSPGQSITISCTGTGSDVGSYNLVSWYQQHPGKAPKLMIYGDSERPSGVSNRFSGSKSGNTASLTISGLQAEDEADYYCSSYAGSGIYVFGTGTKVTVL

SEQ ID NO: 10 - protein, 116 aa, synthetic construct
  REGION 1..116: note = antibody heavy chain variable region
EVQLLESGGGLVQPGGSLRLSCAASGFTFSTYAMGWVRQAPGKGLEWVSSIGASGSQTRYADSVKGRFTISRDNSKNTLYLQMNSLRAEDTAVYYCARLAIGDSYWGQGTMVTVSS

(19) United States
(12) Patent Application Publication
(10) Pub. No.: US 2026/0242484 A1
(43) Pub. Date: Aug. 20, 2026

(54) COMPOSITIONS AND METHODS FOR TREATING HEMOLYTIC DISEASE OF THE FETUS AND NEWBORN

(71) Applicant: MOMENTA PHARMACEUTICALS, INC., Titusville, NJ (US)

(72) Inventors: Anna BEUTLER, Spring House, PA (US); Jocelyn LEU, Spring House, PA (US); Leona LING, Spring House, PA (US); Waheeda SIRAH, Spring House, PA (US); Yosuke KOMATSU, Spring House, PA (US); Xie XU, San Diego, CA (US); Yan XU, Spring House, PA (US)

(21) Appl. No.: 19/152,540

(22) PCT Filed: Feb. 2, 2024
(86) PCT No.: PCT/US2024/014252

§ 371 Date: Jul. 31, 2025

Related U.S. Application Data
(60) Provisional application No. 63/483,266, filed on Feb. 4, 2023.

(51) Int. Cl. C07K 16/28; A61K 39/00; A61P 7/06

(52) U.S. Cl. CPC C07K 16/283; A61P 7/06; A61K 2039/505; A61K 2039/54; A61K 2039/545

(57) ABSTRACT

Provided herein are methods for treating hemolytic disease of the fetus and newborn using compositions comprising anti-FcRn antibodies.

### CROSS REFERENCE

This application claims the benefit of U.S. Provisional Application 63/483,266, filed on Feb. 4, 2023, which is incorporated by reference herein in its entirety.

### FIELD

The present application is related to treating hemolytic disease of the fetus and newborn (HDFN) in a fetal subject using a composition comprising an anti-FcRn antibody.

### BACKGROUND

Hemolytic disease of the fetus and newborn (HDFN) is a rare and potentially life-threatening condition that occurs when the blood types of the mother and fetus are incompatible, and the mother develops alloantibodies against the antigen on fetal red blood cells (RBCs). During pregnancy, the maternal alloantibodies cross the placenta and bind to fetal RBCs possessing the corresponding antigen, thereby causing RBC destruction and anemia in the fetus or newborn. Maternal alloantibodies to many RBC antigens have been observed, however, the RBC antigen most commonly implicated in severe HDFN (defined as fetal anemia requiring intrauterine transfusion (IUT), or fetal demise or hydrops fetalis as a result of HDFN) is RhD, and, to a lesser frequency, Kell, RhC, Rhc, and RhE. Typically, HDFN disease severity increases and gestational age (GA) of onset decreases with every pregnancy with an antigen-positive fetus, due to repeated alloimmunization (Urbaniak 2000; Lobato 2008; Jacobs 1962), although there are exceptions (Zwiers 2018a). Implementation of programs for antenatal and postnatal anti-D immune globulin prophylaxis has led to a significant reduction in the frequency of RhD alloimmunization (from 16% to ≤0.3%) in RhD-negative women and associated fetal/neonatal complications (Bowman 1985, 1988; Koelewijn 2008; Huchet 1987). However, D alloimmunization has not been completely eliminated because of failure to administer anti-D immune globulin in accordance with published guidelines and because of unsuspected fetomaternal bleeding early in gestation, before administration of third-trimester prophylaxis (Badami 2014). Together with non-RhD alloimmunization, for which no prophylaxis is available, HDFN affects approximately 3-80 per 100,000 patients per year in countries with well-established health care infrastructure and Rh disease immunoprophylaxis (Koelewijn 2009; Delaney 2015).

The clinical presentation of HDFN ranges from mild to life-threatening. While mildly affected neonates may require only postnatal phototherapy for jaundice (Ree 2017), fetal anemia in severe HDFN cases requires intervention to prevent development of fetal hydrops (severe edema in the skin and serous cavities). In such cases, fetal erythropoiesis is unable to compensate for the loss of RBCs, and enlargement of the fetal spleen and liver may occur, along with hyperdynamic circulation, causing cardiomegaly and congestive heart failure. If the fetal anemia is untreated, fetal demise is almost certain. There is no approved drug for the treatment of severe HDFN. In current standard of care practice, HDFN pregnancies at risk for fetal anemia are monitored frequently by fetal middle cerebral artery (MCA)-Doppler (Mari 2000; Oepkes 2006). Intrauterine transfusion (IUT) of matched donor blood is performed if the peak systolic velocity (PSV) of blood flow is ≥1.5 multiples of the median (MoM) for GA and cordocentesis confirms moderate to severe fetal anemia. In a large retrospective analysis of all IUTs for RBC alloimmunization performed from 1988 to 2015 in the Netherlands, an average of 3 (range 1-7) IUTs per fetus were required over the course of the pregnancy to treat fetal anemia. (Zwiers 2017). An IUT is a resource-intensive procedure carried out with trained personnel (maternal fetal medicine specialist, anesthesiologist, specially trained nurses and technicians) in the operating room at major medical centers. Thus, there remains an urgent unmet medical need for an effective nonsurgical intervention to treat pregnant women at risk for severe HDFN. The embodiments provided for herein fulfil these needs as well as others.

### DRAWINGS

FIG. 1A is a graph showing the proportion of participants with live birth at or after GA Week 32 and without an IUT throughout entire pregnancies among each regimen group in UNITY study. FIG. 1B is a graph showing proportion of maternal subjects with live birth at or after GA Week 32 and without an IUT throughout entire pregnancies by dose.

FIG. 2 is a graph showing the median serum nipocalimab concentrations in participants among each regimen group in UNITY study.

FIGS. 3A-3D are graphs showing outcomes of nipocalimab administered to mothers at high risk of severe HDFN in UNITY study: FIG. 3A shows proportions of neonates requiring phototherapy; FIG. 3B shows proportions of neonates and infants requiring simple transfusions; FIG. 3C shows mean number of simple transfusions required by neonates and infants; and FIG. 3D shows mean volume of simple transfusions required by neonates and infants.

FIG. 4 is a graph comparing percentage of participants giving live birth at GA≥32 weeks without IUT between UNITY and CLARITY studies.

FIG. 5 is a graph comparing each of the 13 participants' on-study pregnancy (right panel) with a previous qualifying pregnancy (left panel).

### SUMMARY

In some embodiments, a method of treating, or reducing severity of, hemolytic disease of the fetus and newborn (HDFN) in a fetal subject is provided. In some embodiments, the method comprises administering a composition comprising an anti-FcRn antibody to a pregnant woman carrying the fetal subject.

In some embodiments, a method of treating, or reducing severity of, HDFN in a fetal subject, comprising administering a composition comprising nipocalimab to a pregnant woman carrying the fetal subject; wherein nipocalimab is administered intravenously at about 45 mg/kg based on the weight of the pregnant woman; and wherein the composition is administered weekly for at least 20 weeks, and first administration is based on the weight of the pregnant woman on the day of the first administration, and each subsequent administration is based on the weight of the pregnant woman assessed a week or two weeks before the subsequent administration.

### DETAILED DESCRIPTION

The present disclosure provides for embodiments for compositions and methods of treating, or reducing severity of, hemolytic disease of the fetus and newborn (HDFN) and related symptoms or pathologies associated with the same in a fetal subject. The method comprises administering an antibody against human neonatal Fc receptor (FcRn) to a pregnant woman carrying the fetal subject. The antibody against human FcRn can be referred to as an “anti-FcRn antibody.” The anti-FcRn antibodies disclosed herein are useful, e.g., to promote clearance of autoantibodies in a subject, to suppress antigen presentation in a subject, to block an immune response (e.g., block an immune complex-based activation of the immune response in a subject), and/or to treat immunological diseases (e.g., autoimmune diseases or alloimmune diseases) in a subject.

In some embodiments, the anti-FcRN antibody is nipocalimab, which can also be referred to as M281. Nipocalimab is an antibody that binds to FcRn and is described, for example, in U.S. Pat. No. 10,676,526, PCT Publication No. WO2020/023310, and PCT Publication No. WO2020/018910, all of which are hereby incorporated by reference in their entireties.

The term “antibody” herein is used in the broadest sense and encompasses various antibody structures, including but not limited to monoclonal antibodies, polyclonal antibodies, multispecific antibodies (e.g., bispecific antibodies), scFVs, nanobodies, VHH, and antibody fragments so long as they exhibit FcRn antigen-binding activity.

Additionally, antibody or antibody molecule, as that term is used herein, refers to a polypeptide, e.g., an immunoglobulin chain or fragment thereof, comprising at least one functional immunoglobulin variable domain sequence. An antibody molecule encompasses antibodies (e.g., full-length antibodies) and antibody fragments. In some embodiments, an antibody molecule comprises an antigen binding or functional fragment of a full-length antibody, or a full-length immunoglobulin chain. For example, a full-length antibody is an immunoglobulin (Ig) molecule (e.g., an IgG antibody) that is naturally occurring or formed by normal immunoglobulin gene fragment recombinatorial processes. In some embodiments, an antibody molecule refers to an immunologically active, antigen-binding portion of an immunoglobulin molecule, such as an antibody fragment. An antibody fragment (e.g., a functional fragment) comprises a portion of an antibody, e.g., Fab, Fab′, F(ab′)₂, F (ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)₂ fragments, and single chain variable fragments (scFvs).

Immunoglobulin chains exhibit the same general structure of relatively conserved framework regions (FRs) joined by three hypervariable regions, also called complementarity determining regions (CDRs). The CDRs from the two chains of each pair are aligned by the framework regions, enabling binding to a specific epitope. From N-terminus to C-terminus, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

The term “antibody molecule” also encompasses whole or antigen binding fragments of domain, or single domain, antibodies, which can also be referred to as “sdAb” or “VHH.” Domain antibodies comprise either VH or VL that can act as stand-alone, antibody fragments. Additionally, domain antibodies include heavy-chain-only antibodies (HCAbs). Domain antibodies also include a CH2 domain of an IgG as the base scaffold into which CDR loops are grafted. It can also be generally defined as a polypeptide or protein comprising an amino acid sequence that is comprised of four framework regions interrupted by three complementarity determining regions. This is represented as FR1-CDR1-FR2-CDR2-FR3-CDR3-FR4. sdAbs can be produced in camelids such as llamas, but can also be synthetically generated using techniques that are well known in the art. The numbering of the amino acid residues of a sdAb or polypeptide is according to the general numbering for VH domains given by Kabat et al. (“Sequence of proteins of immunological interest,” US Public Health Services, NIH Bethesda, MD, Publication No. 91, which is hereby incorporated by reference). According to this numbering, FR1 of a sdAb comprises the amino acid residues at positions 1-30, CDR1 of a sdAb comprises the amino acid residues at positions 31-36, FR2 of a sdAb comprises the amino acids at positions 36-49, CDR2 of a sdAb comprises the amino acid residues at positions 50-65, FR3 of a sdAb comprises the amino acid residues at positions 66-94, CDR3 of a sdAb comprises the amino acid residues at positions 95-102, and FR4 of a sdAb comprises the amino acid residues at positions 103-113. Domain antibodies are also described in International Publication Nos. WO2004/041862 and WO2016/065323, both of which are hereby incorporated by reference in their entireties.

In some embodiments, as provided for herein, antibody molecules can be monospecific (e.g., monovalent or bivalent), bispecific (e.g., bivalent, trivalent, tetravalent, pentavalent, or hexavalent), trispecific (e.g., trivalent, tetravalent, pentavalent, hexavalent), or with higher orders of specificity (e.g., tetraspecific) and/or higher orders of valency beyond hexavalency. An antibody molecule can comprise a functional fragment of a light chain variable region and a functional fragment of a heavy chain variable region, or heavy and light chains may be fused together into a single polypeptide.

As used herein, the terms “variable region” and “variable domain” refer to the portions of the light and heavy chains of an antibody that include amino acid sequences of complementary determining regions (e.g., CDR L1, CDR L2, CDR L3, CDR H1, CDR H2, and CDR H3) and framework regions. According to the methods used in this disclosure, the amino acid positions assigned to CDRs and FRs are defined according to Kabat (“Sequences of Proteins of Immunological Interest”, 5th Ed. Public Health Service, National Institutes of Health, Bethesda, MD. (1991)). Using this numbering system, the actual linear amino acid sequence may contain fewer or additional amino acids corresponding to a shortening of, or insertion into, a CDR (defined further herein) or FR (defined further herein) of the variable region. For example, a heavy chain variable region may include a single inserted residue (i.e., residue 52a according to Kabat) after residue 52 of CDR H2 and inserted residues (i.e., residues 82a, 82b, 82c, etc. according to Kabat) after residue 82 of heavy chain FR. The Kabat numbering of residues may be determined for a given antibody by alignment at regions of homology of the sequence of the antibody with a “standard” Kabat numbered sequence.

As used herein, the terms “complementary determining regions” and “CDRs” refer to the regions of an antibody variable domain or variable region which are hypervariable in sequence and/or form structurally defined loops. A CDR is also known as a hypervariable region. The light chain and heavy chain variable regions each has three CDRs. The light chain variable region contains CDR L1, CDR L2, and CDR L3. The heavy chain variable region contains CDR H1, CDR H2, and CDR H3. Each CDR may include amino acid residues from a complementarity determining region as defined by Kabat, i.e., about residues 24-34 (CDR L1), 50-56 (CDR L2) and 89-97 (CDR L3) in the light chain variable region and about residues 31-35 (CDR H1), 50-65 (CDR H2) and 95-102 (CDR H3) in the heavy chain variable region.

As used herein, the term “FcRn” refers a neonatal Fc receptor that binds to the Fc region of an IgG antibody, e.g., an IgG1 antibody. An exemplary FcRn is human FcRn having UniProt ID No. P55899, which is hereby incorporated by reference in its entirety. Without being bound to any particular theory, human FcRn is believed to be responsible for maintaining the half-life of IgG by binding and trafficking constitutively internalized IgG back to the cell surface for the recycling of IgG.

In some embodiments, the anti-FcRn antibody comprises a heavy chain or light chain. In some embodiments, the anti-FcRN antibody comprises a heavy chain and a light chain in a scFv format. In some embodiments, the heavy and light chain are linked with a peptide linker, such a glycine/serine or glycine/alanine linker.

In some embodiments, the anti-FcRn antibody is M281 (nipocalimab). In some embodiments, nipocalimab comprises or consists of: a light chain comprising or consisting of the sequence:

(SEQ ID NO: 1)
QSALTQPASVSGSPGQSITISCTGTGSDVGSYNLVSWYQ
QHPGKAPKLMIYGDSERPSGVSNRFSGSKSGNTASLTIS
GLQAEDEADYYCSSYAGSGIYVFGTGTKVTVLGQPKAA
PSVTLFPPSSEELQANKATLVCLISDFYPGAVTVAWKAD
SSPVKAGVETTTPSKQSNNKYAASSYLSLTPEQWKSHKS
YSCQVTHEGSTVEKTVAPTECS;

and a heavy chain comprising or consisting of the sequence:

(SEQ ID NO: 2)
EVQLLESGGGLVQPGGSLRLSCAASGFTFSTYAMGWVR
QAPGKGLEWVSSIGASGSQTRYADSVKGRFTISRDNSKN
TLYLQMNSLRAEDTAVYYCARLAIGDSYWGQGTMVTV
SSASTKGPSVFPLAPSSKSTSGGTAALGCLVKDYFPEPVT
VSWNSGALTSGVHTFPAVLQSSGLYSLSSVVTVPSSSLG
TQTYICNVNHKPSNTKVDKKVEPKSCDKTHTCPPCPAPE
LLGGPSVFLFPPKPKDTLMISRTPEVTCVVVDVSHEDPE
VKFNWYVDGVEVHNAKTKPREEQYASTYRVVSVLTVL
HQDWLNGKEYKCKVSNKALPAPIEKTISKAKGQPREPQ
VYTLPPSREEMTKNQVSLTCLVKGFYPSDIAVEWESNG
QPENNYKTTPPVLDSDGSFFLYSKLTVDKSRWQQGNVF
SCSVMHEALHNHYTQKSLSLSPG.

In some embodiments, the anti-FcRn antibody has: (a) a light chain sequence that is at least 95%, 96%, 97% 98%, or 99% identical to SEQ ID NO: 1, wherein the CDR L1 comprises the sequence TGTGSDVGSYNLVS (SEQ ID NO: 3), the CDR L2 comprises the sequence GDSERPS (SEQ ID NO: 4), the CDR L3 comprises the sequence SSYAGSGIYV (SEQ ID NO: 5); and (b) a heavy chain sequence that is at least 95%, 96%, 97% 98%, or 99% identical to SEQ ID NO: 2, wherein the CDR H1 comprises the sequence TYAMG (SEQ ID NO: 6), the CDR H2 comprises the sequence SIGASGSQTRYADS (SEQ ID NO: 7), and the CDR H3 comprises the sequence LAIGDSY (SEQ ID NO: 8).

In some embodiments, the anti-FcRn antibody comprises a light chain variable region comprising an amino acid sequence that is at least 95%, 97%, 99%, or 100% identical to:
QSALTQPASVSGSPGQSITISCTGTGSDVGSYNLVSWYQQHPGKAPKLMIYGDSERPS GVSNRFSGSKSGNTASLTISGLQAEDEADYYCSSYAGSGIYVFGTGTKVTVL (SEQ ID NO: 9). In some embodiments, the light chain variable region contains a CDR L1 having the sequence of SEQ ID NO: 3, a CDR L2 having the sequence of SEQ ID NO: 4, a CDR L3 having the sequence of SEQ ID NO: 5.

In some embodiments, the anti-FcRn antibody comprises a heavy chain variable region comprising an amino acid sequence that is at least 95%, 97%, 99%, or 100% identical to:
EVQLLESGGGLVQPGGSLRLSCAASGFTFSTYAMGWVRQAPGKGLEWVSSIGASGS QTRYADSVKGRFTISRDNSKNTLYLQMNSLRAEDTAVYYCARLAIGDSYWGQGTM VTVSS (SEQ ID NO: 10). In some embodiments, the heavy chain variable region contains a CDR H1 having the sequence of SEQ ID NO: 6, a CDR H2 having the sequence of SEQ ID NO: 7, and a CDR H3 having the sequence of SEQ ID NO: 8.

In some embodiments, some of the heavy chains of nipocalimab may comprise an additional Lysine (K) residue at the C-terminus of SEQ ID NO: 2.

The term “nipocalimab” as used herein is intended to encompass biosimilar anti-FcRn antibodies that are equivalent to the nipocalimab protein developed and marketed by Johnson and Johnson. For example, a biosimilar of nipocalimab may be acceptable to the European Medicines Agency (EMA) and/or the United States Food and Drug Administration (FDA) when it has essentially the same pharmacological effects as the reference product nipocalimab. For example, a biosimilar of nipocalimab may comprise or consist of the same heavy and light chains sequences as nipocalimab, with the exception of a C-terminal modification. For example the C-terminal amino acid or acids of the heavy chain may be absent, e.g. compared to SEQ ID NO: 2. The present invention extends to the use of antibody that uses the International Nonproprietary Name (INN) “nipocalimab”.

In some embodiments, the antibodies may further comprise amino acid substitutions, additions, and/or deletions outside of the CDRs (i.e., in framework regions (FRs)). An amino acid substitution, addition, and/or deletion can be a substitution, addition, and/or deletion of one or more amino acids (e.g., 1, 2, 3, 4, 5, 6, 7, 8, or more). An amino acid substitution, addition, and/or deletion can be a substitution, addition, and/or deletion of eight or fewer, seven or fewer, six or fewer, five or fewer, four or fewer, three or fewer, or two or fewer single amino acids.

In some embodiments, the antibodies described herein may include amino acid substitutions, additions, and/or deletions in the constant regions (e.g., Fc region) of the antibody that, e.g., lead to decreased effector function, e.g., decreased complement-dependent cytolysis (CDC), antibody-dependent cell-mediated cytolysis (ADCC), and/or antibody-dependent cell-mediated phagocytosis (ADCP), and/or decreased B-cell killing. The constant regions are not involved directly in binding an antibody to its target, but exhibit various effector functions, such as participation of the antibody in antibody-dependent cellular toxicity. In some embodiments, the antibodies described herein are characterized by decreased binding (i.e., absence of binding) to human complement factor Clq and/or human Fc receptor on natural killer (NK) cells. In other embodiments, the antibodies are characterized by decreased binding (i.e., absence of binding) to human FcγRI, FcγRIIA, and/or FcγRIIIA. In some embodiments, the antibodies described herein contain asparagine (N) at position 297 (numbering according to the EU System). In some embodiments, an anti-FcRn antibody described herein is aglycosylated at position 297 (numbering according to the EU System). In some cases, an anti-FcRn antibody described herein does not have an N at position 297 (EU numbering) in any one of SEQ ID NOs: 2, such that the antibody is aglycosylated at that position. The resulting effectorless antibody shows very little binding to complement or Fc receptors (i.e., complement Clq binding), indicating low CDC potential. In some embodiments, the Fc region is an effectorless Fc region.

In some embodiments, the heavy chain of the isolated antibody comprises a sequence having no more than 5, 4, 3, 2 or 1 single amino acid substitutions relative to the amino acid sequence of SEQ ID NO: 2. In some embodiments, the light chain of the isolated antibody comprises a sequence having no more than 5, 4, 3, 2 or 1 single amino acid substitutions relative to the sequence of SEQ ID NO: 1.

As used herein, the term “percent (%) identity” refers to the percentage of amino acid (or nucleic acid) residues of a candidate sequence, e.g., an anti-FcRn antibody of the disclosure, that are identical to the amino acid (or nucleic acid) residues of a reference sequence, e.g., a wild-type anti-FcRn antibody, after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximal alignment over the full length of the sequences being compared. In some embodiments, the percent amino acid (or nucleic acid) sequence identity of a given candidate sequence to, with, or against a given reference sequence (which can alternatively be phrased as a given candidate sequence that has or includes a certain percent amino acid (or nucleic acid) sequence identity to, with, or against a given reference sequence) is calculated as follows:

$$100 \times ( fraction of A / B )$$

where A is the number of amino acid (or nucleic acid) residues scored as identical in the alignment of the candidate sequence and the reference sequence, and where B is the total number of amino acid (or nucleic acid) residues in the reference sequence. In some embodiments where the length of the candidate sequence does not equal to the length of the reference sequence, the percent amino acid (or nucleic acid) sequence identity of the candidate sequence to the reference sequence would not equal to the percent amino acid (or nucleic acid) sequence identity of the reference sequence to the candidate sequence.

In some embodiments, a reference sequence aligned for comparison with a candidate sequence may show that the candidate sequence exhibits from 50% to 100% identity across the full length of the candidate sequence or a selected portion of contiguous amino acid (or nucleic acid) residues of the candidate sequence. The length of the candidate sequence aligned for comparison purpose is at least 30%, e.g., at least 40%, e.g., at least 50%, 60%, 70%, 80%, 90%, or 100% of the length of the reference sequence. When a position in the candidate sequence is occupied by the same amino acid (or nucleic acid) residue as the corresponding position in the reference sequence, then the molecules are identical at that position. A position may be altered by a substitution, deletion, or insertion. A substitution, deletion, or insertion may comprise a certain number of amino acids (e.g., 1, 2, 3, 4, 5, 6, 7, 8, 9, 10, 11, 12, 13, 14, 15, or more). When describing a substitution, deletion, or insertion of no more than n amino acids, this is meant that the substitution, deletion, or insertion comprises, e.g., 1, 2, 3, 4, 5, 6, 7, 8, 9, 10, 11, 12, 13, 14, 15, or n amino acids. The number or substitutions, deletions, or insertions can comprise a percent of the total sequence (e.g., 1%, 5%, 10%, 15%, 20%, or more) where the number of substitutions, deletions, or insertions alters 5%, 10%, 15%, 20% or more, of the amino acids in the total sequence.

In some embodiments, the Fc domain of the antibody is not fucosylated. In some embodiments, the Fc domain of the antibody is not glycosylated. In some embodiments, the antibody lacks effector function. In some embodiments of all the methods described herein, the antibody is an IgG1 antibody.

In some embodiments, the anti-FcRn antibody is nipocalimab, RVT-1401 (HL161), rozanolixizumab (UCB7665), ALXN1830, ABY-039, or efgartigimod. RVT-1401 (also referred to as HL161BKN) is described in PCT Publication No. WO2020/097099, rozanolixizumab is described in PCT Publication No. WO2014/019727, and efgartigimod (ARGX-113) is described in PCT Publication No. WO2015/100299, each of which is hereby incorporated by reference in its entirety. In some embodiments, the anti-FcRn antibody is a biosimilar of any of anti-FcRN antibodies provided for herein. In some embodiments, the anti-FcRn antibody comprises the heavy and light chain CDRs of nipocalimab. In preferred embodiments, the anti-FcRn antibody is nipocalimab. In some embodiments, the anti-FcRn antibody is RVT-1401 (HL161). In some embodiments, the anti-FcRn antibody is rozanolixizumab (UCB7665). In some embodiments, the anti-FcRn antibody is ALXN1830. In some embodiments, the anti-FcRn antibody is ABY-039. In some embodiments, the anti-FcRn antibody is efgartigimod.

In some embodiments, the methods described herein comprise administering the anti-FcRn antibody to the pregnant woman carrying the fetal subject. In some embodiments, the antibody is administered as part of a pharmaceutical composition and about 15 mg/kg to 60 mg/kg of the antibody is administered based on the weight of the pregnant subject. In some embodiments, the antibody is administered at a dose from about 15 mg/kg to about 60 mg/kg, about 15 mg/kg to about 45 mg/kg, about 30 mg/kg to about 45 mg/kg, or about 30 mg/kg to about 60 mg/kg. In some embodiments the antibody is administered at a dose of 15 mg/kg. In some embodiments the antibody is administered at a dose of 30 mg/kg. In some embodiments the antibody is administered at a dose of 45 mg/kg. In some embodiments the antibody is administered at a dose of 60 mg/kg. In some embodiments, the antibody is administered at a dose of 15, 20, 25, 30, 35, 40, 45, 50, 55, or 60 mg/kg. A dosage of 45 mg/kg may be preferred.

In some embodiments, the maximum dose may be 5,400 mg. For example, the pharmaceutical composition may be administered at a dose of 45 mg/kg, with the proviso that the dose does not exceed 5,400 mg.

In some embodiments, the calculated dose may be rounded to the nearest 0.1 kg.

In some embodiments, the antibody is administered weekly, once every two weeks, or monthly. Weekly administrations may be preferred. In some embodiments the antibody is administered for at least 20 weeks. In some embodiments the antibody is administered for a maximum of 23 weeks. In some embodiments, the antibody is administered starting from gestation week (GW) 13, GW 14, GW 15, or GW 16. In some embodiments, the antibody is administered until GW 35. In some embodiments, the antibody is administered weekly from GW13 until GW 35. In some embodiments, the antibody is administered weekly from GW 14 until GW 35. In some embodiments, the antibody is administered weekly from GW 15 until GW 35. In some embodiments, the antibody is administered weekly from GW 16 until GW 35. As used herein, the terms “weekly” or “every week” refer to every 7 days (±1 day), and the terms “biweekly” or “every two weeks” refer to every 14 days (±1 day).

In some embodiments, the antibody is administered as part of a pharmaceutical composition and about 15 mg/kg to 60 mg/kg of the antibody is administered based on the pregnant woman's weight. In some embodiments, the dose is based on the weight of the pregnant woman on the day of the first administration. In some embodiments, the first administration is based on the weight of the pregnant woman on the day of the first administration, and each subsequent administration is based on the weight of the pregnant woman assessed a week before the subsequent administration. In some embodiments, the first administration is based on the weight of the pregnant woman on the day of the first administration, and subsequent administrations are based on the weight of the pregnant woman assessed a week before the subsequent administration. In some embodiments, first administration is based on the weight of the pregnant woman on the day of the first administration, and each subsequent administration is based on the weight of the pregnant woman assessed two weeks before the subsequent administration. In some embodiments, first administration is based on the weight of the pregnant woman on the day of the first administration, and subsequent administrations are based on the weight of the pregnant woman assessed two weeks before the subsequent administration. In some embodiments, the administration takes place in about 15 minutes, about 30 minutes, about 45 minutes, about 60 minutes, about 90 minutes, or about 120 minutes.

In some embodiments, the dose of the first administration is based on the weight of the pregnant woman on the day of the first administration, and the doses of subsequent administrations are based on the weight of the pregnant woman assessed on the day of the immediate previous administration. In some embodiments, the dose of the first administration is based on the weight of the pregnant woman on the day of the first administration, and the dose of each subsequent administration is based on the weight of the pregnant woman assessed on the day of the immediate previous administration. “Immediate previous administration” as used herein refers to the administration of the composition or anti-FcRn antibody that took place immediately before the subsequent administration for which a dose is being determined (for example, the administration of the composition or anti-FcRn antibody that took place one week before, in the case of weekly administrations).

In some embodiments, the dose of the first administration is based on the weight of the pregnant woman on the day of the first administration, and the doses of subsequent administrations are based on the weight of the pregnant woman assessed on the day of the immediate previous administration. In some embodiments, the dose of the first administration is based on the weight of the pregnant woman on the day of the first administration, and the dose of each subsequent administration is based on the weight of the pregnant woman assessed on the day of the immediate previous administration. “Immediate previous administration” as used herein refers to the administration of the composition or anti-FcRn antibody that took place immediately before the subsequent administration for which a dose is being determined (for example, the administration of the composition or anti-FcRn antibody that took place one week before, in the case of weekly administrations).

In some embodiments, the composition is administered weekly for at least 20 weeks, and the pregnant woman has a FcRn receptor occupancy of at least 90% after first administration of the composition, and the pregnant woman maintains a FcRn receptor occupancy of at least 90% until after last administration of the composition. The FcRn receptor occupancy may reach 90% or higher any time after the first administration and prior to the second administration. For example, the FcRn receptor occupancy may reach 90% or higher within 1, 2, 3, 4, 5, or 6 days after the first administration. A FcRn receptor occupancy of 90% or higher is considered full receptor occupancy in the industry. FcRn receptor occupancy can be measured using flow cytometry.

The composition may be administered by any suitable route. In some embodiments, the composition is administered intravenously. In some embodiments, the composition is administered by intravenous infusion.

In some embodiments, the pharmaceutical composition comprises one or more of sodium phosphate, sodium chloride, trehalose, or polysorbate.

As used herein, the term “pharmaceutical composition” refers to a medicinal or pharmaceutical formulation that contains an active ingredient as well as one or more excipients and diluents to enable the active ingredient suitable for the method of administration. The pharmaceutical composition of the present disclosure includes, for example, pharmaceutically acceptable components that are compatible with the anti-FcRn antibody. The pharmaceutical composition may be in aqueous form for intravenous administration.

In some embodiments, the pharmaceutical composition that comprises an anti-FcRn antibody as the therapeutic may be formulated for intravenous administration.

As used herein, the term “pharmaceutically acceptable carrier” refers to an excipient or diluent in a pharmaceutical composition. The pharmaceutically acceptable carrier must be compatible with the other ingredients of the formulation and not deleterious to the recipient. In the present disclosure, the pharmaceutically acceptable carrier must provide adequate pharmaceutical stability to the Fc construct. The nature of the carrier differs with the mode of administration. For example, for intravenous administration, an aqueous solution carrier is generally used; for oral administration, a solid carrier is preferred.

As used herein, the term “therapeutically effective amount” refers to an amount (e.g., pharmaceutical dose) effective in inducing a desired biological effect in a subject or patient or in treating a patient having a condition or disorder described herein. It is also to be understood herein that a “therapeutically effective amount” may be interpreted as an amount giving a desired therapeutic effect, either taken in one dose or in any dosage or route, taken alone or in combination with other therapeutic agents.

As used herein, the term “no more than” refers to an amount that is less than equal to. This may be an amount in integers. For example, no more than two substitutions can refer to 0, 1, or 2 substitutions.

As used herein, the terms “treat”, “treatment” or “treating” refer to reducing, decreasing, decreasing the risk of, or decreasing the side effects of a particular disease or condition. Reducing, decreasing, decreasing the risk of, or decreasing the side effects of are relative to a subject who did not receive treatment, e.g., a control, a baseline, or a known control level or measurement.

In some embodiments, methods of treating hemolytic disease of the fetus and newborn (HDFN) in a fetal subject are provided. In some embodiments, the fetal subject is a fetal subject with, or at risk for, HDFN. In some embodiments, the HDFN is severe HDFN. In some embodiments, the fetal subject with, or at risk for, HDFN is an antigen positive fetus. In some embodiments, the fetal subject with, or at risk for, HDFN is an antigen positive fetus, wherein the antigen is RhD, Kell, Rhc, RhE, RhC, or any combination thereof. In some embodiments, the fetal subject with, or at risk for, HDFN is an antigen positive fetus, wherein the antigen is RhD, Kell, Rhc, RhE, RhC, or any combination thereof, and wherein the pregnant woman carrying the fetal subject is antigen negative. In some embodiments, the pregnant woman carrying the fetal subject is antigen negative. In some embodiments, the pregnant woman is antigen negative, wherein the antigen is RhD, Kell, Rhc, RhE, RhC, or any combination thereof. In some embodiments, the fetal subject with, or at risk for, HDFN is an antigen positive fetus, wherein the antigen is RhD, Kell, Rhc, RhE, RhC, or any combination thereof, and wherein the pregnant woman carrying the fetal subject is antigen negative, wherein the antigen is RhD, Kell, Rhc, RhE, RhC, or any combination thereof. In some embodiments, the fetal subject with, or at risk for, HDFN is positive for an antigen selected from the group consisting of RhD, Kell, Rhc, RhE, RhC, or any combination thereof and the pregnant woman carrying the fetal subject is negative for the same antigen. In some embodiments, the fetal subject with, or at risk for, HDFN is a subject whose pregnant mother has alloantibodies to an antigen, wherein the antigen is RhD, Kell, Rhc, RhE, RhC, or any combination thereof. In some embodiments, the fetal subject with, or at risk for, HDFN is positive for an antigen selected from the group consisting of RhD, Kell, Rhc, RhE, RhC, or any combination thereof and the pregnant woman carrying the fetal subject has maternal alloantibodies to the same antigen. In some embodiments, the maternal alloantibodies are of an isotype selected from IgG1, IgG2, IgG3, or IgG4. In some embodiments, the maternal alloantibodies have a titer selected from: greater than or equal to 4 for anti-Kell; greater than or equal to 16 for anti-RhD; greater than or equal to 16 for anti-Rhc; greater than or equal to 16 for anti-RhE; greater than or equal to 16 for anti-RhC; or any combination thereof.

In some embodiments, the pregnant woman carrying the fetal subject is a pregnant woman with obstetrical history. In some embodiments, the obstetrical history comprises previous pregnancy with a fetus, wherein said fetus is: a fetus with fetal anemia requiring IUT; a fetus with fetal hydrops with MCA-PSV MoM greater than or equal to 1.5; or a fetus with HDFN-related stillbirth at less than or equal to 25 weeks gestation, and/or maternal alloantibody titers for RhD, Kell, Rhc, RhE, or RhC antigen above the critical levels (anti-Kell≥4; other≥16). In some embodiments, the pregnant woman has obstetrical history comprising a previous pregnancy with a previous fetus, wherein: i) the woman received one or more intrauterine transfusions (IUTs) to treat fetal anemia during the previous pregnancy; ii) the previous fetus suffered from fetal anemia; and/or iii) the previous pregnancy resulted in fetal demise and the woman had detectable maternal alloantibody titers to one or more of RhD, Kell, Rhc, RhE, or RhC antigens above a critical level during the previous pregnancy, wherein said critical level of anti-Kell maternal alloantibody titer is ≥4, said critical level of anti-RhD maternal alloantibody titer is ≥16, said critical level of anti-Rhc maternal alloantibody titer is ≥16, said critical level of anti-RhE maternal alloantibody titer is ≥16, and said critical level of anti-RhC maternal alloantibody titer is ≥16. In some embodiments, the pregnant woman has obstetrical history comprising a previous pregnancy with a previous fetus, wherein: i) the woman received one or more intrauterine transfusions (IUTs) to treat fetal anemia during the previous pregnancy; ii) the previous fetus suffered from fetal anemia; and iii) the previous pregnancy resulted in fetal demise and the woman had detectable maternal alloantibody titers to one or more RhD, Kell, Rhc, RhE, or RhC antigens above a critical level during the previous pregnancy, wherein said critical level of anti-Kell maternal alloantibody titer is ≥4, said critical level of anti-RhD maternal alloantibody titer is ≥16, said critical level of anti-Rhc maternal alloantibody titer is ≥16, said critical level of anti-RhE maternal alloantibody titer is ≥16, and said critical level of anti-RhC maternal alloantibody titer is ≥16. In some embodiments, the pregnant woman has obstetrical history comprising a previous pregnancy with a previous fetus, wherein: i) the woman received one or more intrauterine transfusions (IUTs) to treat fetal anemia during the previous pregnancy; ii) the previous fetus suffered from fetal anemia; or iii) the previous pregnancy resulted in fetal demise and the woman had detectable maternal alloantibody titers to one or more RhD, Kell, Rhc, RhE, or RhC antigens above a critical level during the previous pregnancy, wherein said critical level of anti-Kell maternal alloantibody titer is ≥4, said critical level of anti-RhD maternal alloantibody titer is ≥16, said critical level of anti-Rhc maternal alloantibody titer is ≥16, said critical level of anti-RhE maternal alloantibody titer is ≥16, and said critical level of anti-RhC maternal alloantibody titer is ≥16 during the previous pregnancy.

In some embodiments, the method comprises administering a pharmaceutical composition comprising an anti-FcRn antibody to a pregnant woman to treat HDFN. In some embodiments, the anti-FcRn antibody is nipocalimab, RVT-1401 (HL161), rozanolixizumab (UCB7665), ALXN1830, ABY-039, or efgartigimod. In some embodiments, the antibody is nipocalimab. In some embodiments, the antibody is RVT-1401 (HL161). In some embodiments, the antibody is rozanolixizumab (UCB7665). In some embodiments, the antibody is ALXN1830. In some embodiments, the antibody is ABY-039. In some embodiments, the antibody is efgartigimod. In some embodiments, the anti-FcRn antibody is as provided for herein. In some embodiments, the method comprises administering a pharmaceutical composition comprising an anti-FcRn antibody to the pregnant woman with, or at risk for, HDFN to treat the fetal subject carried by the pregnant woman.

In some embodiments, the pregnant woman carrying the fetal subject treated for HDFN shows an improvement in one or more of the following assays, scores or criteria, which can be used to evaluate the improvement or condition of the pregnant woman carrying the fetal subject with HDFN. In some embodiments, the pregnant woman carrying the fetal subject shows improvement in one or more of the following: Generalized Anxiety Disorder 7-item (GAD-7) score, 36-Item Short Form Health Survey version 2 Acute (SF-36 v2 Acute) score, EuroQOL five-dimension questionnaire (EQ-5D-5L) score, or any combination thereof. In some embodiments, the pregnant woman carrying the fetal subject has obstetrical history comprising a previous pregnancy with a previous fetus, wherein the fetal subject shows a reduction in severity of HDFN compared with the previous fetus and/or the fetal subject shows an improvement in MCA-PSV MoM score compared with the previous fetus.

In some embodiments, the pregnant woman carrying the fetal subject being treated for HDFN has or shows a reduction in one or more immunoglobulin isotypes or total IgG. In some embodiments, the reduction is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, or 80%. In some embodiments, the isotype of immunoglobulins reduced is IgG1, IgG2, IgG3, IgG4, or any combination thereof.

In some embodiments, the pregnant woman carrying the fetal subject being treated for HDFN has or shows a reduction in pathogenic alloantibodies. In some embodiments, the reduction is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%. In some embodiments, the pathogenic alloantibodies reduced are selected from the group comprising: anti-RhD alloantibodies, anti-Kell alloantibodies, anti-Rhc alloantibodies, anti-RhE alloantibodies, anti-RhC alloantibodies, or any combination thereof.

In some embodiments, methods of treating or ameliorating anemia (e.g. mild anemia, or severe anemia), severe anemia with enlargement of the liver and spleen, hyperbilirubinemia, jaundice, hydrops fetalis, severe hyperbilirubinemia, and/or kernicterus in a fetal subject are provided. In some embodiments, the method comprises administering a pharmaceutical composition comprising administering an anti-FcRn antibody to the pregnant woman carrying the fetal subject. In some embodiments, the anti-FcRN antibody is nipocalimab, RVT-1401 (HL161), rozanolixizumab (UCB7665), ALXN1830, ABY-039, or efgartigimod. In some embodiments, the antibody is nipocalimab. In some embodiments, the antibody is RVT-1401 (HL161). In some embodiments, the antibody is rozanolixizumab (UCB7665). In some embodiments, the antibody is ALXN1830. In some embodiments, the antibody is ABY-039. In some embodiments, the antibody is efgartigimod. In some embodiments, the anti-FcRN antibody is as provided for herein. In some embodiments, the fetal subject has or is at risk for having hemolytic disease of the fetus and newborn.

In some embodiments, the fetal subject being treated for HDFN achieves a change in severity of HDFN after administering the anti-FcRn antibody to the pregnant woman carrying the fetal subject. The severity of HDFN is defined as: i) Severe: fetal and neonatal death, hydrops fetalis, or requiring IUT during pregnancy as a result of HDFN; ii) Moderate: neonatal exchange transfusions required as a result of HDFN but no antenatal treatment for HDFN; iii) Mild: neonatal simple transfusions required due to HDFN within 12 weeks after birth, with or without phototherapy, but no exchange transfusions or antenatal treatment for HDFN; iv) Self-limited: treatment for HDFN not required, or hemolysis that only required phototherapy treatment, and/or HDFN was treated with recombinant EPOs/iron supplements; and v) None: pregnancies not affected by HDFN, with hemoglobin above the lower limit of the age-appropriate normal reference range and bilirubin below the upper limit of age-appropriate normal reference range at birth and within 12 weeks after birth in neonates.

In some embodiments, the fetal subject achieves a change in severity of HDFN following treatment with the anti-FcRn antibody. In some embodiments, the change in severity of HDFN is a change from more severe HDFN to less severe HDFN. In some embodiments, the severity of HDFN is classified as severe, moderate, mild, self-limited, or none. In some embodiments, the change in severity of HDFN is a change from severe to moderate. In some embodiments, the change in severity of HDDFN is a change from severe to mild. In some embodiments, the change in severity of HDFN is a change from severe to self-limited. In some embodiments, the change in severity of HDFN is a change from severe to none. In some embodiments, the change in severity of HDFN is a change from moderate to mild. In some embodiments, the change in severity of HDFN is a change from moderate to self-limited. In some embodiments, the change in severity of HDFN is a change from moderate to none. In some embodiments, the change in severity of HDFN is a change from mild to self-limited. In some embodiments, the change in severity of HDFN is a change from mild to none. In some embodiments, the change in severity of HDFN is a change from self-limited to none. In some embodiments, the fetal subject achieves a change in severity of HDFN that is a change from severe to moderate severity of HDFN following administration of the composition comprising the anti-FcRn antibody. In some embodiments, the fetal subject achieves a change in severity of HDFN that is a change from severe to mild severity of HDFN following administration of the composition comprising the anti-FcRn antibody. In some embodiments, the fetal subject achieves a change in severity of HDFN that is a change from severe to self-limited severity of HDFN following administration of the composition comprising the anti-FcRn antibody. In some embodiments, the fetal subject achieves a change in severity of HDFN that is a change from severe to none severity of HDFN following administration of the composition comprising the anti-FcRn antibody. In some embodiments, the fetal subject achieves a change in severity of HDFN that is a change from moderate to mild severity of HDFN following administration of the composition comprising the anti-FcRn antibody. In some embodiments, the fetal subject achieves a change in severity of HDFN that is a change from moderate to self-limited severity following administration of the composition comprising the anti-FcRn antibody. In some embodiments, the fetal subject achieves a change in severity of HDFN is a change from moderate to none severity of HDFN following administration of the composition comprising the anti-FcRn antibody. In some embodiments, the fetal subject achieves a change in severity of HDFN that is a change from mild to self-limited severity of HDFN following administration of the composition comprising the anti-FcRn antibody. In some embodiments, the fetal subject achieves a change in severity of HDFN is a change from mild to none severity of HDFN following administration of the composition comprising the anti-FcRn antibody. In some embodiments, the fetal subject achieves a change in severity of HDFN is a change from self-limited to none severity of HDFN following administration of the composition comprising the anti-FcRn antibody.

As used herein, “severe HDFN” is defined as fetal anemia requiring intrauterine transfusion (IUT), or fetal demise or hydrops fetalis as a result of HDFN.

In some embodiments, the pregnant woman carrying the fetal subject being treated for HDFN does not require intrauterine transfusion (IUT) during pregnancy after being treated with the antibody. In some embodiments, the treatment of HDFN in the fetal subject results in no IUT during pregnancy. In some embodiments, the pregnant woman carrying the fetal subject being treated for HDFN does not experience fetal loss due to HDFN after being treated with the antibody. In some embodiments, the treatment of HDFN in a fetal subject results in no fetal loss due to HDFN. In some embodiments, the treatment of HDFN in a fetal subject results in livebirth of the fetal subject. In some embodiments, the fetal subject is liveborn and the pregnant woman received no IUT before GW 24. In some embodiments, the fetal subject is liveborn at or after GW 32. In some embodiments, the fetal subject is liveborn at or after GW 32 and the pregnant woman received no IUT during pregnancy of the fetal subject. In some embodiments, the treatment of HDFN in a fetal subject results in no neonatal death postpartum due to HDFN. In some embodiments, the treatment of HDFN in a fetal subject results in livebirth of the neonate. In some embodiments, the treatment of HDFN in a fetal subject results in a livebirth of the fetal subject. In some embodiments, the fetal subject is alive for at least 1, 2, 3, 4, 5, 6, 7, 8, 9, 10, 11, 12, 13, 14, 15, 16, 17, 18, 19, 20, 21, 22, 23, or 24 weeks postpartum. In some embodiments, the fetal subject is alive for at least 24 weeks postpartum. In some embodiments, the pregnant woman carrying the fetal subject being treated for hemolytic disease of the fetus and newborn does not experience complications due to hydrops fetalis during pregnancy after being treated with the antibody. In some embodiments, the fetal subject does not have hydrops fetalis. In some embodiments, the treatment of HDFN in a fetal subject results in no complications due to hydrops fetalis during pregnancy. In some embodiments, the treatment prevents complications due to hydrops fetalis during pregnancy/gestation. In some embodiments, the pregnant woman carrying the fetal subject being treated for hemolytic disease of the fetus and newborn does not experience: fetal loss due to HDFN; complications due to hydrops fetalis during pregnancy; and intrauterine transfusion during pregnancy after being treated with the antibody. In some embodiments, the treatment of HDFN in a fetal subject results in: no fetal loss due to HDFN or no neonatal death postpartum due to HDFN; no complications due to hydrops fetalis during pregnancy; and no intrauterine transfusion during pregnancy or no neonatal exchange transfusion postpartum. In some embodiments, the pregnant woman carrying the fetal subject being treated for hemolytic disease of the fetus and newborn experiences reduced occurrence of IUT after being treated with the antibody. In some embodiments, the treatment of HDFN reduces the occurrence of hydrops fetalis. In some embodiments, the pregnant woman has obstetrical history comprising a previous pregnancy with a previous fetus, and the pregnant women receives fewer IUT compared with the previous pregnancy. In some embodiments, the fetal subject does not receive an exchange transfusion within 12 weeks following birth. In some embodiments, the fetal subject does not receive a simple transfusion within 12 weeks following birth.

In some embodiments, the pregnant woman has obstetrical history comprising a previous pregnancy with a previous fetus. In some embodiments, the fetus had or was diagnosed with HDFN. In some embodiments, the HFDN was sever HDFN.

In some embodiments, the pregnant woman carrying the fetal subject being treated for HDFN achieves a change from baseline in GAD-7 score on GAD-7 scale after being treated with the antibody. The GAD-7 scale is a self-administered questionnaire that is designed to measure anxiety (Spitzer et al, “A brief measure for assessing generalized anxiety disorder: the GAD-7”, Arch Intern Med. 2006 May 22; 166 (10): 1092-7; Löwe et al., “Validation and standardization of the Generalized Anxiety Disorder Screener (GAD-7) in the general population”, Med Care. 2008 March; 46 (3): 266-74, the content of which are incorporated herein by reference). The recall period for all items is the past 2 weeks. Responses to all items are rated on a 4-point Likert scale ranging from 0 “not at all” to 3 “nearly every day”. The total score ranges from 0 to 21, with higher scores indicating higher severity of anxiety symptoms. The GAD-7 can generally be completed in 2-3 minutes.

In some embodiments, the pregnant woman carrying the fetal subject achieves a change from baseline in GAD-7 score on GAD-7 scale over time following administration of the composition comprising the anti-FcRN antibody. In some embodiments, the change from baseline in GAD-7 score indicates improvement. In some embodiments, the improvement is at least 5%, 10%, 15%, 20%, 25%, 30%, 40%, 50%, 60%, 70%, 80%, 90%, 100%, 150%, 200%, 250%, or 300% as compared to the baseline.

In some embodiments, the pregnant woman carrying the fetal subject being treated for HDFN achieves a change from baseline in SF-36 v2 Acute score on SF-36 v2 Acute scale after being treated with the antibody. The SF-36 Health Survey version 2 is a self-administered, 36-item questionnaire measuring health-related quality of life (HRQOL) (Maruish ME (Ed.) (2011): User's Manual for the SF-36v2 Health Survey. QualityMetric Inc. 3rd ed. Lincoln, RI: QualityMetric Inc., the content of which are incorporated herein by reference). The recall period for all items is the past week. The SF-36 version 2 Acute includes 8 domains that measure physical functioning, role limitations due to physical health problems, bodily pain, general health, vitality social functioning, role limitations due to emotional problems, and mental health. The 8 domains can be aggregated into 2 summary scales that reflect physical and mental health: a physical component summary and a mental component summary. Responses to all items are rated on a 3—, 5- or 6-point Likert scale, with higher scores indicating better health status. The SF-36 version 2 Acute can generally be completed in 5-10 minutes.

In some embodiments, the pregnant woman carrying the fetal subject achieves a change from baseline in SF-36 v2 Acute score on SF-36 v2 Acute scale over time following administration of the composition comprising the anti-FcRn antibody. In some embodiments, the pregnant woman carrying the fetal subject achieves a change from baseline in physical component summary (PCS) SF-36 v2 Acute score on SF-36 v2 Acute scale over time following administration of the composition comprising the anti-FcRn antibody. In some embodiments, the pregnant woman carrying the fetal subject achieves a change from baseline in mental component summary (MCS) SF-36 v2 Acute score on SF-36 v2 Acute scale over time following administration of the composition comprising the anti-FcRn antibody. In some embodiments, the pregnant woman carrying the fetal subject achieves a change from baseline in physical component summary (PCS) and mental component summary (MCS) SF-36 v2 Acute scores on SF-36 v2 Acute scale over time following administration of the composition comprising the anti-FcRn antibody. In some embodiments, the change from baseline in SF-36 v2 Acute score indicates improvement. In some embodiments, the change from baseline in PCS SF-36 v2 Acute score indicates improvement. In some embodiments, the change from baseline in MCS SF-36 v2 Acute score indicates improvement. In some embodiments, the change from baseline in PCS and MCS SF-36 v2 Acute scores indicates improvement. In some embodiments, the improvement is at least 5%, 10%, 15%, 20%, 25%, 30%, 40%, 50%, 60%, 70%, 80%, 90%, 100%, 150%, 200%, 250%, or 300% as compared to the baseline.

In some embodiments, the pregnant woman carrying the fetal subject being treated for HDFN achieves a change from baseline in EQ-5D-5L score on EQ-5D-5L scale after being treated with the anti-FcRn antibody. The EQ-5D-5L Descriptive System is a self-administered, standardized measure of health status in a wide range of health conditions and treatments (Herdman et al., “Development and preliminary testing of the new five-level version of EQ-5D (EQ-5D-5L)”, Qual Life Res. 2011 December; 20 (10): 1727-36; Janssen et al., “Measurement properties of the EQ-5D-5L compared to the EQ-5D-3L across eight patient groups: a multi-country study”, Qual Life Res. 2013 September; 22 (7): 1717-27, the content of which are incorporated herein by reference). It provides a descriptive profile and a single index value for health status that can be used in the clinical and economic evaluation of health care. The recall period for all items is ‘Today’. The EQ-5D-5L consists of the EQ-5D descriptive system and the EQ visual analogue scale (EQ-VAS). The EQ-5D descriptive system is comprised of 5 items across the following 5 dimensions: mobility, self-care, usual activities, pain/discomfort and anxiety/depression. The EQ-5D-5L uses a 5-point Likert response scale ranging from “No problems” to “Extreme problems”, with higher scores indicating better quality of life. The EQ-5D health states defined by the EQ-5D descriptive system can be converted into a single index value using country-specific value sets. The index value facilitates the calculation of quality-adjusted life years that are used to inform economic evaluations of health care interventions. The EQ-5D also includes a VAS that has endpoints labeled “best imaginable health state” and “worst imaginable health state” anchored at 100 and 0, respectively. Participants are asked to indicate how they rate their own health by indicating the point on the EQ-VAS which best represents their own health on that day. The EQ-5D-5L can generally be completed in 2-3 minutes.

In some embodiments, the pregnant woman carrying the fetal subject achieves a change from baseline in EQ-5D-5L score on EQ-5D-5L scale over time following administration of the composition comprising the anti-FcRN antibody. In some embodiments, the pregnant woman carrying the fetal subject achieves a change from baseline in EQ-5D score on EQ-5D scale over time following administration of the composition comprising the anti-FcRN antibody. In some embodiments, the pregnant woman carrying the fetal subject achieves a change from baseline in VAS score on VAS scale over time following administration of the composition comprising the anti-FcRN antibody. In some embodiments, the change from baseline in EQ-5D-5L score indicates improvement. In some embodiments, the change from baseline in EQ-5D score indicates improvement. In some embodiments, the change from baseline in VAS score indicates improvement. In some embodiments, the improvement is at least 5%, 10%, 15%, 20%, 25%, 30%, 40%, 50%, 60%, 70%, 80%, 90%, 100%, 150%, 200%, 250%, or 300% as compared to the baseline.

In some embodiments, the pregnant woman carrying the fetal subject being treated for HDFN achieves a change from baseline in middle cerebral artery peak systolic velocity multiples of median (MCA-PSV MoM) score on MCA-PSV MoM scale after being treated with the anti-FcRn antibody. The fetal subject is monitored for anemia via weekly assessment of the MCA PSV via Doppler ultrasonography from screening to birth as per Mari (Mari et al, “Noninvasive Diagnosis by Doppler Ultrasonography of Fetal Anemia Due to Maternal Red-Cell Alloimmunization”, The New England Journal of Medicine, 342:9-14, 2000, the contents of which is incorporated by reference). As per the current standard of care, if the MCA PSV is ≥1.5 MoM for the estimated GA of the fetal subject, a cordocentesis to ascertain fetal anemia should be completed using the following guidelines: i) If the MCA PSV is ≥1.8 MoM, the cordocentesis should be done within 24 hours of measurement; ii) If the MCA PSV is 1.5-1.8 MoM, the investigator may either choose to perform cordocentesis within 24 hours of first measurement based on other clinical considerations or choose to repeat the MCA PSV within 48 hours of the first measurement to confirm the result. If the repeat MCA PSV is ≥1.5, then cordocentesis should be done within 24 hours of the repeat measurement. If the repeat MCA PSV is <1.5, cordocentesis is not necessary. Weekly measurements of the MCA PSV are to be resumed however the investigator may choose to monitor the MCA PSV more frequently than weekly and proceed with cordocentesis if any repeat MCA PSV is ≥1.5; iii) Since other aspects of clinical decision making around criteria for cordocentesis cannot be completely standardized, if the investigator does not specifically follow the above criteria for timing of cordocentesis, the reason for varying from the criteria should be explained in detail in the electronic case report form (eCRF).

In some embodiments, the pregnant woman carrying the fetal subject achieves a change from baseline in MCA-PSV MoM score on MCA-PSV MoM scale over time following administration of the composition comprising the anti-FcRn antibody. In some embodiments, the change from baseline in MCA-PSV MoM score indicates improvement. In some embodiments, the improvement is at least 5%, 10%, 15%, 20%, 25%, 30%, 40%, 50%, 60%, 70%, 80%, 90%, 100%, 150%, 200%, 250%, or 300% as compared to the baseline.

In some embodiments, the treatment of HDFN in the fetal subject results in the neonate achieving improved IQI score on the IQI scale following administration of the composition comprising the anti-FcRn antibody. The IQI is a 7-item proxy-reported (often by a parent or caregiver) mobile questionnaire designed to assess HRQOL by measuring health status in an infant's first year of life (Krabbe 2020; Jabrayilov 2018). Parents and/or caregivers are asked to choose whether the hypothetical child's health is better or worse than the health of their own child. The IQI consists of 7 health attributes including: sleeping, feeding, breathing, stooling/poo, mood, skin, and interaction. Responses to all items are rated on a 4-point Likert scale, with higher scores indicating better quality of life. IQI health states can be used to calculate a utility score. The IQI can be generally completed in 2-3 minutes.

In some embodiments, the treatment of HDFN in the fetal subject results in the neonate achieving improved IQI score on the IQI scale following administration of the composition comprising the anti-FcRN antibody. In some embodiments, the change from baseline in IQI score indicates improvement. In some embodiments, the improvement is at least 5%, 10%, 15%, 20%, 25%, 30%, 40%, 50%, 60%, 70%, 80%, 90%, 100%, 150%, 200%, 250%, or 300% as compared to the baseline.

In some embodiments, the treatment of HDFN in the fetal subject results in no neonatal death in neonates born later than or within gestation week 24. In some embodiments, the treatment of HDFN in the fetal subject results in no IgG placental transfer to the fetal subject. In some embodiments, the treatment of HDFN in the fetal subject blocks IgG placental transfer to the fetal subject. In some embodiments, the treatment of HDFN in the fetal subject results in the neonate showing reduced maternal alloantibodies over time. In some embodiments, the maternal alloantibodies are pathogenic alloantibodies. In some embodiments, the pathogenic alloantibodies are to an antigen selected from RhD, Kell, Rhc, RhE, RhC, or any combination thereof. In some embodiments, the reduction is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%. In some embodiments, the reduction is at least 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%. In some embodiments, the reduction is at least 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold. In some embodiments, the reduction is about 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold.

In some embodiments, after first administration of the anti-FcRn antibody, the concentration of the maternal alloantibodies in the pregnant woman is reduced by at least 8-fold compared to before the first administration. The at least 8-fold reduction in the concentration of the maternal alloantibodies in the pregnant woman may be achieved any time after the first administration and prior to the second administration, e.g., within 1, 2, 3, 4, 5, or 6 days after the first administration. In some embodiments, after the first administration of the antibody the concentration of the maternal alloantibodies in the pregnant woman is reduced by at least 8-fold compared to before first administration of the, and the pregnant woman maintains reduced concentration of the maternal alloantibodies until after last administration of the antibody. In some embodiments, after the first administration of the antibody, the concentration of serum IgG in the pregnant woman is reduced by at least 80% compared to before first administration, and the pregnant woman maintains reduced concentration of serum IgG until after last administration of the antibody. The at least 80% reduction in the concentration of serum IgG in the pregnant woman may be achieved any time after the first administration and prior to the second administration, e.g., within 1, 2, 3, 4, 5, or 6 days after the first administration. The maintenance of reduced concentration of serum IgG until after the last administration of the composition may be, for example, at least 1 day after the last administration of the composition.

In some embodiments, the pregnant woman does not receive intravenous immune globulin (IVIG) prior to last administration of the composition.

As provided for herein, in some embodiments, pharmaceutical compositions comprising an anti-FcRn antibody are provided. In some embodiments, pharmaceutical compositions comprising an anti-FcRn antibody for administration to the pregnant woman carrying the fetal subject suffering from, or at risk for, hemolytic disease of the fetus and newborn wherein the anti-FcRn antibody is administered to the pregnant woman in a therapeutically effective amount from about 15 mg/kg to about 60 mg/kg every week are provided. In some embodiments, the pharmaceutical composition is administered to the pregnant woman carrying the fetal subject in a therapeutically effective amount of about 45 mg/kg every week. In some embodiments, the pregnant woman is pregnant with an antigen positive fetus. In some embodiments, the antigen is RhD, Kell, Rhc, RhE, RhC, or any combination thereof. In some embodiments, the pregnant woman carrying the fetal subject is a pregnant woman with alloantibodies to an antigen. In some embodiments, the antigen is RhD, Kell, Rhc, RhE, RhC, or any combination thereof. In some embodiments, the alloantibody titers are: greater than or equal to 4 for anti-Kell; greater than or equal to 16 for anti-RhD; greater than or equal to 16 for anti-Rhc; greater than or equal to 16 for anti-RhE; greater than or equal to 16 for anti-RhC; or any combination thereof. In some embodiments, the pregnant woman carrying the fetal subject has obstetrical history. In some embodiments, the obstetrical history comprises previous pregnancy with a fetus, wherein said fetus is: a fetus with fetal anemia requiring IUT; a fetus with fetal hydrops with MCA-PSV MoM greater than or equal to 1.5; or a fetus with HDFN-related stillbirth at less than or equal to 25 weeks gestation, and/or maternal alloantibody titers for RhD, Kell, Rhc, RhE, or RhC antigen above the critical levels (anti-Kell≥4; other≥16). In some embodiments, the pregnant woman received no IUT during pregnancy with the fetal subject. In some embodiments, the fetal subject being carried by the pregnant woman is a fetal subject with, or at risk for, severe HDFN.

In some embodiments, a method of treating, or reducing severity of, HDFN in a fetal subject, is provided, wherein the method comprises administering a composition comprising nipocalimab to a pregnant woman carrying the fetal subject; wherein nipocalimab is administered at about 45 mg/kg based on the weight of the pregnant woman; wherein the composition is administered weekly for at least 20 weeks, and first administration is based on the weight of the pregnant woman on the day of the first administration, and each of subsequent administration is based on the weight of the pregnant woman assessed one week or two weeks before the subsequent administration. In some embodiments, a method of treating, or reducing severity of, HDFN in a fetal subject, is provided, wherein the method comprises administering a composition comprising nipocalimab to a pregnant woman carrying the fetal subject; wherein nipocalimab is administered at about 45 mg/kg based on the weight of the pregnant woman; wherein the composition is administered weekly for at least 20 weeks, and first administration is based on the weight of the pregnant woman on the day of the first administration, and subsequent administrations are based on the weight of the pregnant woman assessed one week or two weeks before the subsequent administration.

In some embodiments, the administration of the pharmaceutical composition to the pregnant woman to treat HDFN results in the pregnant woman showing improvement in severity of HDFN, Generalized Anxiety Disorder 7-item (GAD-7) score, 36-Item Short Form Health Survey version 2 Acute (SF-36 v2 Acute) score, EuroQOL five-dimension questionnaire (EQ-5D-5L) score, or any combination thereof. In some embodiments, the administration of the pharmaceutical composition to the pregnant woman to treat hemolytic disease of the fetus and newborn reduces severity of HDFN.

In some embodiments, the administration of the pharmaceutical composition to the pregnant woman to treat HDFN in the fetal subject results in the neonate achieving an improvement in one or more of the following assays, scores or criteria, which can be used to evaluate the improvement or condition of the neonate. In some embodiments, the neonate shows improvement in Infant Health-Related Quality of Life Instrument (IQI) score.

In some embodiments, the administration of the pharmaceutical composition to the pregnant woman carrying the fetal subject shows a reduction in one or more immunoglobulin isotypes or total IgG in the pregnant woman. In some embodiments, the isotype is IgG1, IgG2, IgG3, or IgG4. In some embodiments, the reduction is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, or 80%.

In some embodiments, the administration of the pharmaceutical composition to the pregnant woman carrying the fetal subject shows a reduction in pathogenic alloantibodies. In some embodiments, the reduction is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%. In some embodiments, the pathogenic alloantibodies reduced are selected from the group comprising: RhD, Kell, Rhc, RhE, RhC, or any combination thereof.

In some embodiments, the administration of the pharmaceutical composition to the pregnant woman carrying the fetal subject treats or ameliorates anemia (e.g. mild anemia or severe anemia), severe anemia with enlargement of the liver and spleen, hyperbilirubinemia, jaundice, hydrops fetalis, severe hyperbilirubinemia, or kernicterus, or combinations thereof.

In some embodiments, the administration of the pharmaceutical composition the pregnant woman carrying the fetal subject achieves a change in severity of HDFN following treatment with the anti-FcRn antibody. In some embodiments, the change in severity of HDFN is a change from more severe HDFN to less severe HDFN. In some embodiments, the severity of HDFN is classified as severe, moderate, mild, self-limited, or none. In some embodiments, the change in severity of HDFN is a change from severe to moderate. In some embodiments, the change in severity of HDFN is a change from severe to mild. In some embodiments, the change in severity of HDFN is a change from severe to self-limited. In some embodiments, the change in severity of HDFN is a change from severe to none. In some embodiments, the change in severity of HDFN is a change from moderate to mild. In some embodiments, the change in severity of HDFN is a change from moderate to self-limited. In some embodiments, the change in severity of HDFN is a change from moderate to none. In some embodiments, the change in severity of HDFN is a change from mild to self-limited. In some embodiments, the change in severity of HDFN is a change from mild to none. In some embodiments, the change in severity of HDFN is a change from self-limited to none. In some embodiments, the administration of the pharmaceutical composition to the pregnant woman carrying the fetal subject achieves a change in severity of HDFN that is a change from severe to moderate severity of HDFN following administration of the composition comprising the anti-FcRn antibody. In some embodiments, the administration of the pharmaceutical composition to the pregnant woman carrying the fetal subject achieves a change in severity of HDFN that is a change from severe to mild severity of HDFN following administration of the composition comprising the anti-FcRn antibody. In some embodiments, the administration of the pharmaceutical composition to the pregnant woman carrying the fetal subject achieves a change in severity of HDFN that is a change from severe to self-limited severity of HDFN following administration of the composition comprising the anti-FcRn antibody. In some embodiments, the administration of the pharmaceutical composition to the pregnant woman carrying the fetal subject achieves a change in severity of HDFN that is a change from severe to none severity of HDFN following administration of the composition comprising the anti-FcRn antibody. In some embodiments, the administration of the pharmaceutical composition to the pregnant woman carrying the fetal subject achieves a change in severity of HDFN that is a change from moderate to mild severity of HDFN following administration of the composition comprising the anti-FcRn antibody. In some embodiments, the administration of the pharmaceutical composition to the pregnant woman carrying the fetal subject achieves a change in severity of HDFN that is a change from moderate to self-limited severity following administration of the composition comprising the anti-FcRn antibody. In some embodiments, the administration of the pharmaceutical composition to the pregnant woman carrying the fetal subject achieves a change in severity of HDFN is a change from moderate to none severity of HDFN following administration of the composition comprising the anti-FcRn antibody. In some embodiments, the administration of the pharmaceutical composition to the pregnant woman carrying the fetal subject achieves a change in severity of HDFN that is a change from mild to self-limited severity of HDFN following administration of the composition comprising the anti-FcRn antibody. In some embodiments, the administration of the pharmaceutical composition to the pregnant woman carrying the fetal subject achieves a change in severity of HDFN is a change from mild to none severity of HDFN following administration of the composition comprising the anti-FcRn antibody. In some embodiments, the administration of the pharmaceutical composition to the pregnant woman carrying the fetal subject achieves a change in severity of HDFN is a change from self-limited to none severity of HDFN following administration of the composition comprising the anti-FcRn antibody.

In some embodiments, the administration of the pharmaceutical composition to the pregnant woman carrying the fetal subject being treated for hemolytic disease of the fetus and newborn results in the pregnant woman not requiring intrauterine transfusion during pregnancy after being treated with the pharmaceutical composition. In some embodiments, the administration of the pharmaceutical composition to treat HDFN in the fetal subject results in no intrauterine transfusion during pregnancy. In some embodiments, the administration of the pharmaceutical composition to treat HDFN in the fetal subject results in no neonatal exchange transfusion postpartum. In some embodiments, the administration of the pharmaceutical composition to the pregnant woman carrying the fetal subject being treated for HDFN results in the pregnant woman not experiencing fetal loss due to HDFN after being treated with the antibody. In some embodiments, the administration of the pharmaceutical composition to treat HDFN in the fetal subject results in no fetal loss due to HDFN. In some embodiments, the administration of the pharmaceutical composition to treat HDFN in the fetal subject results in no neonatal death postpartum due to HDFN. In some embodiments, the administration of the pharmaceutical composition to the pregnant woman carrying the fetal subject being treated for HDFN results in the pregnant woman not experiencing complications due to hydrops fetalis during pregnancy after being treated with the antibody. In some embodiments, the administration of the pharmaceutical composition to treat HDFN in the fetal subject results in no complications due to hydrops fetalis during pregnancy. In some embodiments, the administration of the pharmaceutical composition to the pregnant woman carrying the fetal subject being treated for HDFN results in the pregnant woman not experiencing: fetal loss due to HDFN; complications due to hydrops fetalis during pregnancy; and intrauterine transfusion during pregnancy after being treated with the antibody. In some embodiments, the treatment of HDFN in the fetal subject results in: no fetal loss due to HDFN or no neonatal death postpartum due to HDFN; no complications due to hydrops fetalis during pregnancy; and no intrauterine transfusion during pregnancy or no neonatal exchange transfusion postpartum. In some embodiments, the administration of the pharmaceutical composition to the pregnant woman carrying the fetal subject being treated for HDFN results in the pregnant woman experiencing reduced occurrence of intrauterine transfusions after being treated with the anti-FcRn antibody. In some embodiments, the administration of the pharmaceutical composition to treat HDFN reduces the occurrence of intrauterine transfusions. In some embodiments, the administration of the pharmaceutical composition to treat HDFN reduces the occurrence of hydrops fetalis. In some embodiments, the administration of the pharmaceutical composition to treat HDFN reduces the occurrence of neonatal exchange transfusions. In some embodiments, the administration of the pharmaceutical composition to treat HDFN reduces the occurrence of simple transfusions within 12 weeks following birth.

In some embodiments, the administration of the pharmaceutical composition to the pregnant woman carrying the fetal subject being treated for hemolytic disease of the fetus and newborn achieves a change from baseline in GAD-7 score on GAD-7 scale over time following administration of the composition comprising the anti-FcRn antibody. In some embodiments, the change from baseline in GAD-7 score indicates improvement. In some embodiments, the improvement is at least 5%, 10%, 15%, 20%, 25%, 30%, 40%, 50%, 60%, 70%, 80%, 90%, 100%, 150%, 200%, 250%, or 300% as compared to the baseline.

In some embodiments, the administration of the pharmaceutical composition to the pregnant woman carrying the fetal subject being treated for hemolytic disease of the fetus and newborn achieves a change from baseline in SF-36 v2 Acute score on SF-36 v2 Acute scale over time following administration of the composition comprising the anti-FcRn antibody. In some embodiments, the administration of the pharmaceutical composition to the pregnant woman carrying the fetal subject being treated for HDFN achieves a change from baseline in physical component summary (PCS) SF-36 v2 Acute score on SF-36 v2 Acute scale over time following administration of the composition comprising the anti-FcRn antibody. In some embodiments, the administration of the pharmaceutical composition to the pregnant woman carrying the fetal subject being treated for HDFN achieves a change from baseline in mental component summary (MCS) SF-36 v2 Acute score on SF-36 v2 Acute scale over time following administration of the composition comprising the anti-FcRn antibody. In some embodiments, the administration of the pharmaceutical composition to the pregnant woman carrying the fetal subject being treated for HDFN achieves a change from baseline in physical component summary (PCS) and mental component summary (MCS) SF-36 v2 Acute scores on SF-36 v2 Acute scale over time following administration of the composition comprising the anti-FcRn antibody. In some embodiments, the change from baseline in SF-36 v2 Acute score indicates improvement. In some embodiments, the change from baseline in PCS SF-36 v2 Acute score indicates improvement. In some embodiments, the change from baseline in MCS SF-36 v2 Acute score indicates improvement. In some embodiments, the change from baseline in PCS and MCS SF-36 v2 Acute scores indicates improvement. In some embodiments, the improvement is at least 5%, 10%, 15%, 20%, 25%, 30%, 40%, 50%, 60%, 70%, 80%, 90%, 100%, 150%, 200%, 250%, or 300% as compared to the baseline.

In some embodiments, the administration of the pharmaceutical composition to the pregnant woman carrying the fetal subject being treated for hemolytic disease of the fetus and newborn achieves a change from baseline in EQ-5D-5L score on EQ-5D-5L scale over time following administration of the composition comprising the anti-FcRN antibody. In some embodiments, the administration of the pharmaceutical composition to the pregnant woman carrying the fetal subject being treated for HDFN achieves a change from baseline in EQ-5D score on EQ-5D scale over time following administration of the composition comprising the anti-FcRn antibody. In some embodiments, the administration of the pharmaceutical composition to the pregnant woman carrying the fetal subject being treated for HDFN achieves a change from baseline in VAS score on VAS scale over time following administration of the composition comprising the anti-FcRn antibody. In some embodiments, the change from baseline in EQ-5D-5L score indicates improvement. In some embodiments, the change from baseline in EQ-5D score indicates improvement. In some embodiments, the change from baseline in VAS score indicates improvement. In some embodiments, the improvement is at least 5%, 10%, 15%, 20%, 25%, 30%, 40%, 50%, 60%, 70%, 80%, 90%, 100%, 150%, 200%, 250%, or 300% as compared to the baseline.

In some embodiments, the administration of the pharmaceutical composition to the pregnant woman carrying the fetal subject being treated for hemolytic disease of the fetus and newborn achieves a change from baseline in MCA-PSV MoM score on MCA-PSV MoM scale over time following administration of the composition comprising the anti-FcRn antibody. In some embodiments, the change from baseline in MCA-PSV MoM score indicates improvement. In some embodiments, the improvement is at least 5%, 10%, 15%, 20%, 25%, 30%, 40%, 50%, 60%, 70%, 80%, 90%, 100%, 150%, 200%, 250%, or 300% as compared to the baseline.

In some embodiments, the administration of the pharmaceutical composition to the pregnant woman carrying the fetal subject being treated for hemolytic disease of the fetus and newborn results in the neonate achieving improved IQI score on the IQI scale following administration of the composition comprising the anti-FcRn antibody. In some embodiments, the change from baseline in IQI score indicates improvement. In some embodiments, the improvement is at least 5%, 10%, 15%, 20%, 25%, 30%, 40%, 50%, 60%, 70%, 80%, 90%, 100%, 150%, 200%, 250%, or 300% as compared to the baseline.

In some embodiments, the administration of the pharmaceutical composition to the pregnant woman carrying the fetal subject being treated for hemolytic disease of the fetus and newborn results in no neonatal death in neonates born later than or within gestation week 24. In some embodiments, the administration of the pharmaceutical composition to the pregnant woman carrying the fetal subject being treated for HDFN results in no IgG placental transfer to the fetus. In some embodiments, the administration of the pharmaceutical composition to the pregnant woman carrying the fetal subject being treated for HDFN blocks IgG placental transfer to the fetus. In some embodiments, the administration of the pharmaceutical composition to the pregnant woman carrying the fetal subject being treated for HDFN results in neonate showing reduced maternal alloantibodies over time. In some embodiments, the maternal alloantibodies are pathogenic alloantibodies. In some embodiments, the pathogenic alloantibodies are to an antigen selected from RhD, Kell, Rhc, RhE, RhC, or any combination thereof. In some embodiments, the reduction is about 1%, 2%, 3%, 4%, 5%, 6%, 7%, 8%, 9%, 10%, 1-20%, 5-20%, 5-25%, 10-30%, 15-35%, 20-40%, 40-60%, or, about, or at least, 5%, 10%, 15%, 20%, 25%, 30%, 35%, 40%, 45%, 50%, 60%, 70%, 80%, 90%, or 95%.

As used herein, the term “change” in reference to a baseline refers to a subject having an improvement as compared to that subject's conditions, scores, symptoms, and the like prior to being treated with the anti-FcRn antibodies as provided for herein.

“Baseline” refers to a subject prior to being treated with a therapeutic, such as an anti-FcRn antibody, including those provided for herein.

As provided herein, in some embodiments, the maternal/fetal improvement in symptoms or conditions are referred to as occurring 22 weeks after initiation of treatment. As provided herein, in some embodiments, the maternal improvement in symptoms or conditions are referred to as occurring 22 weeks after initiation of treatment and 24 weeks following delivery. As provided herein, in some embodiments, the neonate/infant/child improvement in symptoms or conditions are referred to as occurring 96 weeks after birth. Although reference is made to determining whether those improvements are measurable at 22, 24, and 96 weeks, in some embodiments, the improvements or changes described herein occur within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, and 96 weeks. In some embodiments, the changes or improvements last at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, and 96 weeks.

In some embodiments, the pregnant woman carrying the fetal subject being treated for hemolytic disease of the fetus and newborn with an anti-FcRn antibody has received concomitant or previous treatment comprising: i) Has previously received nipocalimab; ii) Has previously received batoclimab, efgartigimod, rozanolixizumab, or other anti-FcRn investigational agents; iii) Is currently receiving systemic corticosteroids or other immunosuppressants for disorders unrelated to the pregnancy (Note: use of low-potency topical corticosteroids, nasal/inhaled corticosteroids, or intra-articular corticosteroids is permitted); iv) Has received or planning to receive plasmapheresis, immunoadsorption therapy, IVIg, or any IgG Fc-related protein therapeutics during the current pregnancy; v) Has received a live vaccine within 3 months prior to screening or has a known need to receive a live vaccine during the study or within at least 3 months after the last administration of study intervention in this study; or vi) Has previously received rituximab or eculizumab within 6 months prior to screening.

In some embodiments, the pregnant woman carrying the fetal subject has previously been treated with nipocalimab, batoclimab, efgartigimod, rozanolixizumab, corticosteroids, immunosuppressants, plasmapheresis, immunoadsorption therapy, IVIG, rituximab, eculizumab, RhoGAM, blood transfusion, intravenous fluids, exchange transfusion, intrauterine transfusion, or any combination thereof. In some embodiments, the pregnant woman carrying the fetal subject has been previously treated with a first anti-FcRn antibody and is then switched to a different anti-FcRn antibody. In some embodiments, the pregnant woman carrying the fetal subject has been previously treated with a therapeutic to treat HDFN that is not nipocalimab.

In some embodiments, the pregnant woman carrying the fetal subject is treated with an additional therapeutic or therapy in addition to the anti-FcRn antibody. In some embodiments, the additional therapeutic or therapy is RhoGAM, blood transfusion, intravenous fluids, exchange transfusion, intrauterine transfusion, IVIG, plasmapheresis, immunoadsorption therapy, or any combination thereof. In some embodiments, the additional therapeutic or therapy is RhoGAM. In some embodiments, the additional therapeutic or therapy is blood transfusion. In some embodiments, the additional therapeutic or therapy is intravenous fluids. In some embodiments, the additional therapeutic or therapy is exchange transfusion. In some embodiments, the additional therapeutic or therapy is intrauterine transfusion. In some embodiments, the additional therapeutic or therapy is IVIG. In some embodiments, the additional therapeutic or therapy is plasmapheresis. In some embodiments, the additional therapeutic or therapy is immunoadsorption therapy. In some embodiments, the additional therapeutic is administered concurrently or sequentially (prior to or after) with the anti-FcRn antibody.

In some embodiments, the pharmaceutical composition is co-administered with at least one additional therapeutic or therapy. In some embodiments, the additional therapeutic or therapy is RhoGAM, blood transfusion, intravenous fluids, exchange transfusion, intrauterine transfusion, IVIG, plasmapheresis, immunoadsorption therapy, or any combination thereof. In some embodiments, the additional therapeutic or therapy is RhoGAM. In some embodiments, the additional therapeutic or therapy is blood transfusion. In some embodiments, the additional therapeutic or therapy is intravenous fluids. In some embodiments, the additional therapeutic or therapy is exchange transfusion. In some embodiments, the additional therapeutic or therapy is intrauterine transfusion. In some embodiments, the additional therapeutic or therapy is IVIG. In some embodiments, the additional therapeutic or therapy is plasmapheresis. In some embodiments, the additional therapeutic or therapy is immunoadsorption therapy. In some embodiments, the additional therapeutic is administered concurrently or sequentially (prior to or after) with the anti-FcRn antibody.

In some embodiments, the pregnant woman carrying the fetal subject being treated for HDFN with an anti-FcRn antibody does not experience significantly increased levels of total cholesterol, high-density lipoprotein (HDL), calculated low-density lipoprotein (LDL), and triglycerides after being treated with the antibody. In some embodiments, the antibody is M281 (nipocalimab). In some embodiments, the pregnant woman carrying the fetal subject being treated for HDFN with an anti-FcRn antibody does not experience significantly increased levels of total cholesterol after being treated with the antibody. In some embodiments, the pregnant woman carrying the fetal subject being treated for HDFN with an anti-FcRn antibody does not experience significantly increased levels of high-density lipoprotein (HDL) after being treated with the antibody. In some embodiments, the pregnant woman carrying the fetal subject being treated for HDFN with an anti-FcRn antibody does not experience significantly increased levels of calculated low-density lipoprotein (LDL) after being treated with the antibody. In some embodiments, the pregnant woman carrying the fetal subject being treated for HDFN with an anti-FcRn antibody does not experience significantly increased levels of triglycerides after being treated with the antibody. In some embodiments, the pregnant woman carrying the fetal subject being treated for HDFN with an anti-FcRn antibody does not experience significantly increased levels of total cholesterol after being treated with M281 (nipocalimab). In some embodiments, the pregnant woman carrying the fetal subject being treated for HDFN with an anti-FcRn antibody does not experience significantly increased levels of high-density lipoprotein (HDL) after being treated with M281 (nipocalimab). In some embodiments, the pregnant woman carrying the fetal subject being treated for HDFN with an anti-FcRn antibody does not experience significantly increased levels of calculated low-density lipoprotein (LDL) after being treated with M281 (nipocalimab). In some embodiments, the pregnant woman carrying the fetal subject being treated for HDFN with an anti-FcRn antibody does not experience significantly increased levels of triglycerides after being treated with M281 (nipocalimab).

In some embodiments, the administration of the pharmaceutical composition to the pregnant woman carrying the fetal subject does not significantly increase levels of total cholesterol, high-density lipoprotein (HDL), calculated low-density lipoprotein (LDL), and triglycerides after administration of the pharmaceutical composition comprising the antibody. In some embodiments, the antibody is M281 (nipocalimab). In some embodiments, the administration of the pharmaceutical composition to the pregnant woman carrying the fetal subject does not significantly increase levels of total cholesterol after administration of the pharmaceutical composition comprising the antibody. In some embodiments, the administration of the pharmaceutical composition to the pregnant woman carrying the fetal subject does not significantly increase levels of high-density lipoprotein (HDL) after administration of the pharmaceutical composition comprising the antibody. In some embodiments, the administration of the pharmaceutical composition to the pregnant woman carrying the fetal subject does not significantly increase levels of calculated low-density lipoprotein (LDL) after administration of the pharmaceutical composition comprising the antibody. In some embodiments, the administration of the pharmaceutical composition to the pregnant woman carrying the fetal subject does not significantly increase levels of triglycerides after administration of the pharmaceutical composition comprising the antibody. In some embodiments, the administration of the pharmaceutical composition to the pregnant woman carrying the fetal subject does not significantly increase levels of total cholesterol after administration of the pharmaceutical composition comprising M281 (nipocalimab). In some embodiments, the administration of the pharmaceutical composition to the pregnant woman carrying the fetal subject does not significantly increase levels of high-density lipoprotein (HDL) after administration of the pharmaceutical composition comprising M281 (nipocalimab). In some embodiments, the administration of the pharmaceutical composition to the pregnant woman carrying the fetal subject does not significantly increase levels of calculated low-density lipoprotein (LDL) after administration of the pharmaceutical composition comprising M281 (nipocalimab). In some embodiments, the administration of the pharmaceutical composition to the pregnant woman carrying the fetal subject does not significantly increase levels of triglycerides after administration of the pharmaceutical composition comprising M281 (nipocalimab).

As used herein, the phrase “does not significantly increase” when used in reference to levels (measurements) of total cholesterol, high-density lipoprotein (HDL), calculated low-density lipoprotein (LDL), or triglycerides” means that any increase is less than 25% as compared to the level(s) prior (baseline) to the administration of the antibody or compositions provided for herein. In some embodiments, the increase is less than 25%, 20%, 19%, 18%, 17%, 16%, 15%, 14%, 13%, 12%, 11%, 10%, 9%, 8%, 7%, 6%, or 5%. In some embodiments, the increase is no greater than about 1 to about 30%, about 5% to about 25%, about 1% to about 20%, about 1% to about 15%, about 1% to about 10%, about 5% to about 15%, about 5% to about 20%, about 10% to about 20%, about 1%, about 5%, about 6%, about 7%, about 8%, about 9%, about 10%, about 11%, about 12%, about 13% m about 14%, about 15%, about 16%, about 17%, about 18%, about 19%, about 20%, or about 25%. In some embodiments, the subject being treated for HDFN is a subject in need thereof.

The present invention includes uses, such as medical uses, corresponding to all of the methods disclosed herein, including all optional and/or preferred features of such methods. For example, the present invention provides a composition comprising an anti-FcRn antibody, for use in a method of treating or reducing severity of hemolytic disease of the fetus and newborn (HDFN) in a fetal subject, the method comprising administering the composition to a pregnant woman carrying the fetal subject. The present invention also provides a composition comprising nipocalimab for use in a method of treating orreducing severity of HDFN in a fetal subject, the method comprising administering the composition to a pregnant woman carrying the fetal subject; wherein nipocalimab is administered intravenously at about 45 mg/kg based on the weight of the pregnant woman; and wherein the composition is administered weekly for at least 20 weeks, and first administration is based on the weight of the pregnant woman on the day of the first administration, and each subsequent administration is based on the weight of the pregnant woman assessed a week or two weeks before the subsequent administration. The present invention also provides use of a composition comprising an anti-FcRn antibody in the manufacture of a medicament for treating or reducing severity of hemolytic disease of the fetus and newborn (HDFN) in a fetal subject.

As used herein and in the appended claims, the singular forms “a”, “an” and “the” include plural reference unless the context clearly dictates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by +10% and remain within the scope of the disclosed embodiments.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any composition or method that recites the term “comprising” should also be understood to also describe such compositions as consisting, consisting of, or consisting essentially of the recited components or elements.

As used herein, the term “individual,” “subject,” or “patient,” can be used interchangeably, means any animal, including mammals, such as mice, rats, other rodents, rabbits, dogs, cats, swine, cattle, sheep, horses, or primates, such as humans. As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the phrase “in need thereof” means that the subject has been identified as having a need for the particular method or treatment. In some embodiments, the identification can be by any means of diagnosis. In any of the methods and treatments described herein, the subject can be in need thereof. In some embodiments, the subject is in an environment or will be traveling to an environment in which a particular disease, disorder, or condition is prevalent.

As used herein, the phrase “integer from X to Y” means any integer that includes the endpoints. For example, the phrase “integer from 1 to 5” means 1, 2, 3, 4, or 5.

The following examples are illustrative, but not limiting, of the compounds, compositions and methods described herein. Other suitable modifications and adaptations known to those skilled in the art are within the scope of the following embodiments.

### Enumerated Embodiments

1. A composition comprising an anti-FcRn antibody for use in treating, or reducing severity of, hemolytic disease of the fetus and newborn (HDFN) in a fetal subject, wherein the composition is administered to a pregnant woman carrying the fetal subject. 2. The use of embodiment 1, wherein the fetal subject is antigen positive.
3. The use of embodiments 1 or 2, wherein the pregnant woman is antigen negative. 4. The use of embodiment 2 or 3, wherein the antigen is RhD, Kell, Rhc, RhE, RhC, or any combination thereof.
5. The use of embodiment 3 or 4, wherein the pregnant woman has maternal alloantibodies to the antigen.
6. The use of embodiment 5, wherein the maternal alloantibodies are an isotype selected from IgG1, IgG2, IgG3, or IgG4.
7. The use of embodiment 5 or 6, wherein the maternal alloantibodies have a titer selected from:
greater than or equal to 4 for anti-Kell;
greater than or equal to 16 for anti-RhD;
greater than or equal to 16 for anti-Rhc;
greater than or equal to 16 for anti-RhE;
greater than or equal to 16 for anti-RhC; or any combination thereof.
   8. The use of any one of the preceding embodiments, wherein the fetal subject has a reduction in maternal alloantibodies or total immunoglobulin (IgG) following administration of the composition.
   9. The use of any one of the preceding embodiments, wherein the pregnant woman has obstetrical history comprising a previous pregnancy with a previous fetus, wherein:
i) the woman received one or more intrauterine transfusions (IUTs) to treat fetal anemia during the previous pregnancy;
ii) the previous fetus suffered from fetal anemia; and/or
iii) the previous pregnancy resulted in fetal demise and the woman had detectable maternal alloantibody titers to one or more of RhD, Kell, Rhc, RhE, or RhC antigens above a critical level during the previous pregnancy, wherein said critical level of anti-Kell maternal alloantibody titer is ≥4, said critical level of anti-RhD maternal alloantibody titer is ≥16, said critical level of anti-Rhc maternal alloantibody titer is ≥16, said critical level of anti-RhE maternal alloantibody titer is ≥16, and said critical level of anti-RhC maternal alloantibody titer is ≥16.
   10. The use of any one of the preceding embodiments, wherein the fetal subject is a fetus with or at risk for severe HDFN.
   11. The use of any one of the preceding embodiments, wherein the anti-FcRn antibody is administered at about 15 mg/kg to about 60 mg/kg based on the weight of the pregnant woman.
   12. The use of embodiment 11, wherein the anti-FcRn antibody is administered at about 15 mg/kg to about 30 mg/kg, about 30 mg/kg to about 60 mg/kg, about 45 mg/kg to about 60 mg/kg, about 15 mg/kg to about 45 mg/kg, or about 30 mg/kg to about 45 mg/kg based on the weight of the pregnant woman.
   13. The use of embodiments 11 or 12, wherein the anti-FcRn antibody is administered at about 45 mg/kg based on the weight of the pregnant woman.
   14. The use of any one of embodiments 11-13, wherein the composition is administered weekly for at least 20 weeks, and first administration is based on the weight of the pregnant woman on the day of the first administration, and each of subsequent administration is based on the weight of the pregnant woman assessed a week before the subsequent administration.
   15. The use of any one of embodiments 11-13, wherein the composition is administered weekly for at least 20 weeks, and first administration is based on the weight of the pregnant woman on the day of the first administration, and subsequent administrations are based on the weight of the pregnant woman assessed a week before the subsequent administration.
   16. The use of any one of embodiments 11-13, wherein the composition is administered weekly for at least 20 weeks, and first administration is based on the weight of the pregnant woman on the day of the first administration, and each of subsequent administration is based on the weight of the pregnant woman assessed two weeks before the subsequent administration.
   17. The use of any one of embodiments 11-13, wherein the composition is administered weekly for at least 20 weeks, and first administration is based on the weight of the pregnant woman on the day of the first administration, and subsequent administrations are based on the weight of the pregnant woman assessed two weeks before the subsequent administration.
   18. The use of any one of the preceding embodiments, wherein the composition is administered weekly for a minimum of 20 weeks, and a maximum of 23 weeks.
   19. The use of any one of the preceding embodiments, wherein the composition is administered weekly from gestational week 13, week 14, or week 15.
   20. The use of any one of the preceding embodiments, wherein the composition is administered weekly until gestational week 35.
   21. The use of any one of the preceding embodiments, wherein the composition is administered weekly until delivery.
   22. The use of any one of the preceding embodiments, wherein the composition is administered weekly for at least 20 weeks, and the pregnant woman has a FcRn receptor occupancy of at least 90% after first administration of the composition, and the pregnant woman maintains a FcRn receptor occupancy of at least 90% until after las administration of the composition.
   23. The use of any one of the preceding embodiments, wherein the pregnant woman has obstetrical history comprising a previous pregnancy with a previous fetus.
   24. The use of any one of the preceding embodiments, wherein the pregnant woman has obstetrical history comprising a previous pregnancy with a previous fetus, and the fetal subject shows a reduction in severity of HDFN compared with the previous fetus.
   25. The use of any one of the preceding embodiments, wherein the pregnant woman has obstetrical history comprising a previous pregnancy with a previous fetus, and the fetal subject shows an improvement in MCA-PSV MoM score compared with the previous fetus.
   26. The use of any one of the preceding embodiments, wherein the pregnant woman carrying the fetal subject shows an improvement in one or more assessments selected from: Generalized Anxiety Disorder 7-item (GAD-7) score, 36-Item Short Form Health Survey version 2 Acute (SF-36 v2 Acute) score, EuroQOL five-dimension questionnaire (EQ-5D-5L) score, or combinations thereof.
   27. The use of any one of preceding embodiments wherein the use treats or ameliorates one or more symptoms in the fetal subject selected from: mild anemia, severe anemia, severe anemia with enlargement of the liver and spleen, hyperbilirubinemia, jaundice, hydrops fetalis, severe hyperbilirubinemia, kernicterus, or combinations thereof.
   28. The use of any one of preceding embodiments, wherein the pregnant woman receives no intrauterine transfusion (IUT) during pregnancy of the fetal subject.
   29. The use of any one of preceding embodiments, wherein the fetal subject in liveborn.
   30. The use of any one of preceding embodiments, wherein the fetal subject is liveborn and the pregnant woman received no IUT before gestational week 24.
   31. The use of any one of preceding embodiments, wherein the fetal subject is liveborn at or after gestational week 32 and the pregnant woman received no IUT during pregnancy of the fetal subject.
   32. The use of any one of preceding embodiments, wherein the fetal subject is liveborn at or after gestational week 34.
   33. The use of any one of the preceding embodiments, wherein the use reduces severity of HDFN in the fetal subject following administration of the composition.
   34. The use of any one of the preceding embodiments, wherein the fetal subject does not have hydrops fetalis.
   35. The use of any one of the preceding embodiments, wherein the pregnant woman has obstetrical history comprising a previous pregnancy with a previous fetus, and the pregnant woman received fewer IUTs compared with the previous pregnancy.
   36. The use of any one of the preceding embodiments, wherein the fetal subject does not receive an exchange transfusion within 12 weeks following birth.
   37. The use of any one of the preceding embodiments, wherein the fetal subject does not receive a simple transfusion within 12 weeks following birth.
   38. The use of any one of embodiments 5-37, wherein the composition is administered weekly for at least 20 weeks, after first administration concentration of the maternal alloantibodies in the pregnant woman is reduced by at least 8-fold compared to before first administration of the composition, and optionally the pregnant woman maintains reduced concentration of the maternal alloantibodies until after last administration of the composition.
   39. The use of any one of the preceding embodiments, wherein the composition is administered weekly for at least 20 weeks, after first administration concentration of serum IgG in the pregnant woman is reduced by at least 80% compared to before first administration of the composition, and the pregnant woman maintains reduced concentration of serum IgG until after last administration of the composition.
   40. The use of any one of the preceding embodiments, wherein the use does not significantly increase levels of total cholesterol, HDL, calculated LDL, and triglycerides in the pregnant woman as compared to the levels prior to the administration of the composition.
   41. The use of embodiment 40, wherein the use does not significantly increase levels of total cholesterol in the pregnant woman.
   42. The use of embodiment 40, wherein the use does not significantly increase levels of total HDL in the pregnant woman.
   43. The use of embodiment 40, wherein the use does not significantly increase levels of calculated LDL in the pregnant woman.
   44. The use of embodiment 40, wherein the use does not significantly increase levels of triglycerides in the pregnant woman.
   45. The use of any one of the preceding embodiments, wherein the composition is administered intravenously.
   46. The use of any one of the preceding embodiments, wherein the composition is administered by intravenous infusion.
   47. The use of any one of the preceding embodiments, wherein the pregnant woman does not receive intravenous immune globulin (IVIG) prior to last administration of the composition.
   48. The use of any one of the preceding embodiments, wherein the use further comprises administering an additional therapeutic or therapy to the pregnant woman, and optionally wherein the additional therapeutic or therapy is selected from RhoGAM, blood transfusion, intravenous fluids, exchange transfusion, intrauterine transfusion, IVIG, plasmapheresis, immunoadsorption therapy, or any combination thereof
   49. The use of embodiments 47 or 48, wherein the additional therapeutic or therapy is administered concurrently or sequentially (prior to or after) with the composition.
   50. The use of any one of the preceding embodiments, wherein the anti-FcRn antibody comprises:
(a) a light chain, wherein the CDR L1 comprises the sequence TGTGSDVGSYNLVS (SEQ ID NO: 3), the CDR L2 comprises the sequence GDSERPS (SEQ ID NO: 4), the CDR L3 comprises the sequence SSYAGSGIYV (SEQ ID NO: 5); and
(b) a heavy chain, wherein the CDR H1 comprises the sequence TYAMG (SEQ ID NO: 6), the CDR H2 comprises the sequence SIGASGSQTRYADS (SEQ ID NO: 7), and the CDR H3 comprises the sequence LAIGDSY (SEQ ID NO: 8).
   51. The use of any one of the preceding embodiments, wherein the anti-FcRn antibody comprises a light chain variable region comprising an amino acid sequence of SEQ ID NO: 9.
   52. The use of any one of the preceding embodiments, wherein the anti-FcRn antibody comprises a heavy chain variable region comprising an amino acid sequence of SEQ ID NO: 10.
   53. The use of any one of the preceding embodiments, wherein the anti-FcRn antibody comprises:
a light chain comprising the sequence of SEQ ID NO: 1; and
a heavy chain comprising the sequence of SEQ ID NO: 2.
   54. The use of any one of the embodiments 1-53, wherein the anti-FcRn antibody is nipocalimab.
   55. The use of any one of the preceding embodiments, wherein the anti-FcRn antibody comprises:
(a) a light chain sequence that is at least 95%, 96%, 97% 98%, or 99% identical to SEQ ID NO: 1, wherein the CDR L1 comprises the sequence of SEQ ID NO: 3, the CDR L2 comprises the sequence of SEQ ID NO: 4, the CDR L3 comprises the sequence of SEQ ID NO: 5; and
(b) a heavy chain sequence that is at least 95%, 96%, 97% 98%, or 99% identical to SEQ ID NO: 2, wherein the CDR H1 comprises the sequence of SEQ ID NO: 6, the CDR H2 comprises the sequence of SEQ ID NO: 7, and the CDR H3 comprises the sequence of SEQ ID NO: 8.
   56. The use of any one of the preceding embodiments, wherein the anti-FcRn antibody comprises a light chain variable region comprising an amino acid sequence that is at least 95%, 97%, 99%, or 100% identical to SEQ ID NO: 9.
   57. The use of embodiment 56, wherein the light chain variable region contains a CDR L1 having the sequence of SEQ ID NO: 3, a CDR L2 having the sequence of SEQ ID NO: 4, and a CDR L3 having the sequence of SEQ ID NO: 5.
   58. The use of any one of the preceding embodiments, wherein the anti-FcRn antibody comprises a heavy chain variable region comprising an amino acid sequence that is at least 95%, 97%, 99%, or 100% identical to SEQ ID NO: 10.
   59. The use of embodiment 58, wherein the heavy chain variable region contains a CDR H1 having the sequence of SEQ ID NO: 6, a CDR H2 having the sequence of SEQ ID NO: 7, and a CDR H3 having the sequence of SEQ ID NO: 8.
   60. A composition comprising nipocalimab for use in treating, or reducing severity of, HDFN in a fetal subject, wherein the composition is intravenously administered to a pregnant woman carrying the fetal subject;
wherein nipocalimab is administered at about 45 mg/kg based on the weight of the pregnant woman;
wherein the composition is administered weekly for at least 20 weeks, and first administration is based on the weight of the pregnant woman on the day of the first administration, and each of subsequent administration is based on the weight of the pregnant woman assessed a week or two weeks before the subsequent administration.
   61. A composition comprising nipocalimab for use in treating, or reducing severity of, HDFN in a fetal subject, the method comprising administering a composition comprising nipocalimab to a pregnant woman carrying the fetal subject;
wherein nipocalimab is administered intravenously at about 45 mg/kg based on the weight of the pregnant woman; and
wherein the composition is administered weekly for at least 20 weeks, and first administration is based on the weight of the pregnant woman on the day of the first administration, and subsequent administrations are based on the weight of the pregnant woman assessed a week or two weeks before the subsequent administration.
   62. The use of embodiment 60 or 61, wherein the composition is administered weekly from gestational week 13, week 14, or week 15.
   63. The use of any one of embodiment 60-62, wherein the composition is administered weekly for a maximum of 23 weeks.
   64. The use of any one of embodiments 60-63, wherein the pregnant woman has maternal alloantibodies to one or more antigen, and wherein the antigen is RhD, Kell, Rhc, RhE, RhC, or any combination thereof.
   65. The use of embodiment 64, wherein the composition is administered weekly for at least 20 weeks, after first administration concentration of the maternal alloantibodies in the pregnant woman is reduced by at least 8-fold compared to before first administration of the composition, and the pregnant woman maintains reduced concentration of the maternal alloantibodies until after last administration of the composition.
   66. The use of any one of embodiments 60-65, wherein the composition is administered weekly for at least 20 weeks, and the pregnant woman has a FcRn receptor occupancy of at least 90% after first administration of the composition, and the pregnant woman maintains a FcRn receptor occupancy of at least 90% until after last administration of the composition.
   67. The use of any one of the preceding embodiments, wherein the composition is administered weekly for at least 20 weeks, after first administration concentration of serum IgG in the pregnant woman is reduced by at least 80% compared to before first administration of the composition, and the pregnant woman maintains reduced concentration of serum IgG until after last administration of the composition.
   68. The use of any one of embodiments 60-67, wherein the fetal subject is liveborn at or after gestational week 32 and the pregnant woman receives no IUT during pregnancy of the fetal subject.
   69. The use of any one of embodiments 60-68, wherein the fetal subject is liveborn.
   70. The use of any one of embodiments 60-69, wherein the fetal subject is liveborn and the pregnant woman receives no IUT before gestational week 24.
   71. The use of any one of embodiments 60-70, wherein the fetal subject does not have hydrops fetalis.
   72. The use of any one of embodiments 60-71, wherein the pregnant woman does not receive intravenous immune globulin (IVIG) prior to last administration of the composition.
   73. The use of any one of embodiments 60-72, wherein the pregnant woman has obstetrical history comprising a previous pregnancy with a previous fetus, wherein:
i) the woman received one or more intrauterine transfusions (IUTs) to treat fetal anemia during the previous pregnancy;
ii) the previous fetus suffered from fetal anemia; and/or
iii) the previous pregnancy resulted in fetal demise and the woman had detectable maternal alloantibody titers to one or more of RhD, Kell, Rhc, RhE, or RhC antigens above a critical level during the previous pregnancy, wherein said critical level of anti-Kell maternal alloantibody titer is ≥4, said critical level of anti-RhD maternal alloantibody titer is ≥16, said critical level of anti-Rhc maternal alloantibody titer is ≥16, said critical level of anti-RhE maternal alloantibody titer is ≥16, and said critical level of anti-RhC maternal alloantibody titer is ≥16.
   74. A composition comprising an anti-FcRn antibody for use in a method of treating, or reducing severity of, hemolytic disease of the fetus and newborn (HDFN) in a fetal subject, the method comprising administering the composition to a pregnant woman carrying the fetal subject.
   75. The composition for use according to embodiment 74, wherein the method if as define in any one of embodiments 1 to 59.
   76. A composition comprising nipocalimab for use in a method of treating, or reducing severity of, HDFN in a fetal subject, the method comprising administering the composition to a pregnant woman carrying the fetal subject;
wherein nipocalimab is administered intravenously at about 45 mg/kg based on the weight of the pregnant woman; and
wherein the composition is administered weekly for at least 20 weeks, and first administration is based on the weight of the pregnant woman on the day of the first administration, and each subsequent administration is based on the weight of the pregnant woman assessed a week or two weeks before the subsequent administration.
   77. A composition comprising nipocalimab for use in a method of treating, reducing severity of, or preventing HDFN in a fetal subject, the method comprising administering the composition to a pregnant woman carrying the fetal subject;
wherein nipocalimab is administered intravenously at about 45 mg/kg based on the weight of the pregnant woman; and
wherein the composition is administered weekly for at least 20 weeks, and first administration is based on the weight of the pregnant woman on the day of the first administration, and subsequent administrations are based on the weight of the pregnant woman assessed a week or two weeks before the subsequent administration.
   77. The composition for use according to embodiments 76 or 77, wherein the method is as defined in any one of embodiments 62 to 73.

### EXAMPLES

Example 1. Study Design to Evaluate Safety and Efficacy of Nipocalimab in Pregnant Women at High Risk for Early-Onset-Severe Hemolytic Disease of the Fetus and Newborn: UNITY. This was a phase 2 multicenter, open-label study (UNITY; ClinicalTrials.gov Identifier: NCT03842189) in pregnant women at high risk for early onset severe HDFN (EOS-HDFN) based on their obstetrical history. For this study and the following CLARITY study, HDFN disease onset at GA≤24 weeks was defined as EOS-HDFN. The EOS-HDFN population being at greatest risk for severe HDFN. A sample size of approximately 15 pregnant women was planned, and enrollment occurred at multiple study sites specializing in maternal-fetal medicine and the treatment of HDFN worldwide. Total duration of the study was approximately 50 weeks for each pregnant woman entering the study and 96 weeks for each child born during the study. This study included a maternal screening period of up to approximately 6 weeks, a treatment period of approximately 20 weeks, a 24-week postnatal follow-up period for mothers, and a 96-week follow-up period for all neonates/infants. Initiation of nipocalimab occurred at GA 14 weeks to ensure placental FcRn blocking prior to the acceleration of placental transfer of maternal alloantibodies throughout the second trimester. During the trial, four dosing regimens of nipocalimab were evaluated: baseline weight (BLW)-based 30 mg/kg IV weekly (QW), 30-45 mg/kg IV QW, and 45 mg/kg IV QW; and time-adjusted weight (TAW)-based 45 mg/kg IV QW. 2 participants switched dosing from 30 mg/kg BLW to 45 mg/kg BLW and 2 participants switched dosing from 45 mg/kg BLW to 45 mg/kg TAW during the study; otherwise, participants remained in the same dose group during the study. The time-adjusted weight-based dose was recalculated every two weeks according to the maternal body weight. Nipocalimab administration occurred weekly until GA 35 weeks or delivery of the infant, whichever comes first. If delivery did not occur prior to 35 weeks, infants were delivered at 37 weeks. If an IUT was required, nipocalimab was continued until fetal blood analysis at subsequent IUTs reveals predominantly donor adult cells. Pregnant women received IVIG at a dose of 500 mg/kg 48 to 72 hours before delivery, if not contraindicated, as prophylaxis against maternal infection and to allow for placental transfer of IgG. However, if delivery was urgent or emergent, IVIG was given to the mother before delivery or as soon as possible to the neonate after delivery.

During the follow-up period, neonates/infants who were at risk for infection and have total IgG concentration in cord blood <500 mg/dL or had total IgG concentration in cord blood <200 mg/dL without risk factors for infection received IVIG 500 mg/kg. Risk factors for infection in the neonate included intrapartum maternal fever (>38° C.) or clinical signs of chorioamnionitis, spontaneous preterm premature rupture of membranes, maternal group B streptococcus (GBS) colonization, bacteriuria, or UTI, invasive GBS infection in previous child or a neonate requiring umbilical artery catheterization, central line, or other risk factors for infection as identified by the investigator or study personnel. The primary safety endpoint was the maternal and neonatal safety of nipocalimab, including treatment-emergent AEs, serious AEs, AEs of special interest (i.e., infections requiring oral/IV anti-infective agents, and maternal grade ≥3 hypoalbuminemia), need for concomitant therapies, changes in ECG, vital signs, and laboratory parameters monitored up to 24 weeks post-delivery. Fetal safety outcomes included signs of potential fetal growth restriction, and changes in fetal heart rate during infusions. For neonates, AEs, unexpected/unusual childhood illnesses, IgG levels and neurodevelopment was monitored through Week 24 or through Week 96. The primary efficacy endpoint was the proportion of patients with a live birth at or after GA 32 weeks without an IUT throughout the entire pregnancy. Secondary efficacy endpoints included the percentage of live births, percentage of patients at GA 24 weeks without an IUT, GA at first IUT, number of IUTs required, GA at delivery, percentage of patients with fetal hydrops, percentage of neonates requiring phototherapy, percentage of neonates requiring exchange transfusions, percentage of neonates requiring simple transfusions in the first 12 weeks of life, and number of simple transfusions required by the neonate in the first 12 weeks of life. Other secondary outcomes included PD outcomes (i.e., percentage of maternal FcRn RO, and maternal IgG and alloantibody levels) and PK outcomes (i.e., serum concentration of nipocalimab).

Using PK/PD modelling, leveraging data from a completed phase 1 study (ClinicalTrials.gov Identifier: NCT02828046), the inventors selected Nipocalimab dose of 45 mg/kg QW as a maximum dose for this study. The PK/PD modeling result shows that 45 mg/kg with time-adjusted weight dosing could maintain ≥90% participants with full RO (defined at RO ≥90%)≥1 day and >1 week to ensure that drug holidays or other unexpected disruptions in scheduled infusions that may occur do not cause loss of RO or IgG rebound, while the nipocalimab doses <45 mg/kg did not maintain full RO. The time-adjusted weight-based dosing approach was designed for this study due to increases in maternal weight and the corresponding blood volume and rate of IgG placental transfer during pregnancy. IgG placental transfer was GA dependent, being significantly higher in late pregnancy compared to early pregnancy. Based on the same PK/PD modelling, the mean pre-dose nipocalimab exposure following the time-adjusted weight-based dosing approach would be approximately 14.4% and 22.6% higher at GA Weeks 29 and 36, respectively. Therefore, in order to maintain sufficient nipocalimab exposure during pregnancy, time-adjusted weight-based dosing, in which the nipocalimab dose was re-calculated every 2 weeks from baseline based on the mother's weight, was considered necessary, while the <25% increase in mean exposure would not result in a safety concern.

The following inclusion criteria were followed:

1. Able to understand and voluntarily provide written signed informed consent to participate in the study.
2. Female and ≥18 years of age.
3. Pregnant to an estimated GA of 8 to up to 14 weeks.
4. A previous pregnancy with a gestation that included at least one of the following at ≤24 weeks gestation:
(a) Severe fetal anemia, defined as hemoglobin <0.55 MoM for GA (see table below):

| Weeks' Gestation | Median Hemoglobin (g/dL) | 0.55 MoM |
|---|---|---|
| 16 | 10.0 | 5.5 |
| 17 | 10.4 | 5.7 |
| 18 | 10.6 | 5.9 |
| 19 | 10.9 | 6.0 |
| 20 | 11.2 | 6.1 |
| 21 | 11.4 | 6.3 |
| 22 | 11.6 | 6.4 |
| 23 | 11.8 | 6.5 |
| 24 | 12.0 | 6.6 |

(b) Fetal hydrops (ascites) with MCA-PSV MoM≥4
(c) Stillbirth with fetal or placental pathology indicative of HDFN.
   5. Maternal alloantibody titers for anti-D ≥32 or anti-Kell titers ≥4.
   6. Free fetal DNA consistent with an antigen-positive fetus (blood sample drawn from the mother).
   7. Maternal evidence for immunity to measles, mumps, rubella, and varicella, as documented by serologies performed during Screening. If initial serologies are borderline or negative, they may be repeated at a second lab. Alternatively, vaccination records can be used to support evidence of immunity.
   8. Screening IgG and albumin levels within the laboratory normal ranges.
   9. Willing to receive standard of care with IUT, if clinically indicated.
   10. Agree to receive recommended vaccinations as per local standard of care for both mother and child throughout the course of the study.
   11. Willing to forego collection of cord blood for stem cell storage or other non-study purposes.
   12. For mother and neonate, willing to forego participation in another clinical trial of an investigational therapy for the duration of their participation in the current study.
   13. Willing to consent to a 24-week safety follow-up period for the patient and a 96-week safety follow-up period for the neonate/infant.
   14. It is recommended that patients are up-to-date on age-appropriate vaccinations prior to screening as per routine local medical guidelines. For study patients who received locally-approved (and including emergency use-authorized) COVID-19 vaccines recently prior to study entry, follow applicable local vaccine labelling, guidelines, and standards of care for pregnant women receiving immune-targeted therapy when determining an appropriate interval between vaccination and study enrollment.

This open label multicenter study enrolled 13 maternal participants at risk for early onset severe HDFN. One maternal participant was enrolled twice for 2 successive pregnancies; the first pregnancy was electively terminated at GA Week 17 due to a fetal familial genetic disorder unrelated to HDFN. For this participant, data from the first pregnancy were not considered in the efficacy analysis but were considered for exposure, AEs, and clinical abnormalities, and data from the second pregnancy were included in all applicable analyses. The majority of subjects were white (92%) and not Hispanic or Latino (92%); the mean age was 35.8 years, ranging from 28 to 43 years. In total, 6 of 13 (46%) maternal participants completed treatment with nipocalimab. Four (4) (31%) maternal participants discontinued nipocalimab due to an adverse event (AE) (1 [33%] in the 30 mg/kg BLW group, 1 [25%] in the 45 mg/kg BLW group, and 2 [50%] in the 45 mg/kg TAW group).

### Primary Efficacy Endpoint:

Based on the treatment policy estimated analysis of the primary efficacy endpoint, a statistically significant higher proportion (53.8% [7/13]; 95% CI: 25.1%, 80.8%; p-value <0.001) of maternal participants achieved treatment success (live birth at or after GA Week 32 without an IUT through the entire pregnancy) when compared against the historical benchmark of 10% (p-value<0.001). The 45 mg/kg dose groups (BLW and TAW) had a greater proportion of participants who met the primary efficacy endpoint (5 of 8) than the 30 or 30-45 mg/kg BLW dose groups (2 of 5).

### Selected Secondary Efficacy Endpoints:

Based on the FAS analysis (N=13):
12 (92.3%) of maternal participants had a live birth at any time (95% CI: 64.0%, 99.8%)
12 (92.3%) of maternal participants had a live birth, without an IUT before GA Week 24 (95% CI: 64.0%, 99.8%)
Mean GA at any delivery outcome (live birth or fetal loss), with or without IUT, was Week 34\hat{ }6/7 (range: Week 23\hat{ }6/7 to Week 37\hat{ }3/7), with 6 (46.2%) maternal participants having delivery outcome at GA ≥37 weeks
For the 5 maternal participants with live births who had an IUT, the median GA at first IUT was Week 28\hat{ }3/7 (range, Week 24\hat{ }1/7 to Week 31\hat{ }5/7). Median GA at first IUT regardless of pregnancy outcome (live birth or fetal demise [n=6]) was Week 27\hat{ }1/7 (range, Week 22\hat{ }5/7 to Week 31\hat{ }5/7
No maternal participants had a fetus with fetal hydrops (95% CI: 0.0%, 26.5%)
Comparing study pregnancies with qualifying pregnancies, study pregnancies had a higher proportion that resulted in live births (92.3% vs 38.5%), fewer participants who required IUTs (46.2% vs 84.6%), later median age at first IUT (GA Week 27\hat{ }1/7 vs GA Week 20\hat{ }4/7), and later median GA at delivery (GA Week 36\hat{ }4/7 vs GA Week 23\hat{ }6/7). Among qualifying pregnancies, 7 participants had a fetus with hydrops.
In a post-hoc analysis summarizing the number of IUTs in all participants with live births, the mean (SD) number of IUTs was 1.4 (2.0) in the study pregnancy; the mean (SD) number of IUTs was 5.6 (3.3) amongst all live births in the most recent qualifying pregnancy. The median number of IUTs was 0 (range, 0 to 5) in the study pregnancy; the median number of IUTs was 5 (range, 2 to 11) amongst all live births in the most recent qualifying pregnancy.

Based on the Neonates/Infants (N=12) analysis:
11 (91.7%) neonates/infants had at least 1 phototherapy session
In their first 12 weeks, 6 (50.0%) neonates/infants had at least 1 simple transfusion
In their first 12 weeks, 1 (8.3%) neonate/infant had an exchange transfusion (in the 45 mg/kg TAW group)
Eleven of 12 neonates required hospitalization including 9 who required NICU for care related to EOS HDFN, with a median length of hospital stay of 7.0 days (range, 3 to 64). Reasons leading to hospitalization (treatment of HDFN related AEs, for SOC phototherapy, or feeding problems [1 neonate]) resolved in each neonate.
No formal hypothesis testing was performed on these endpoints.

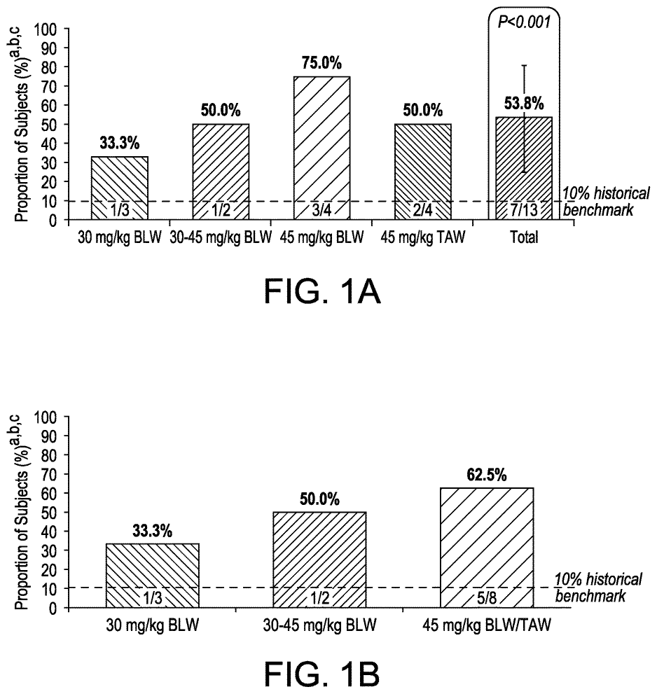

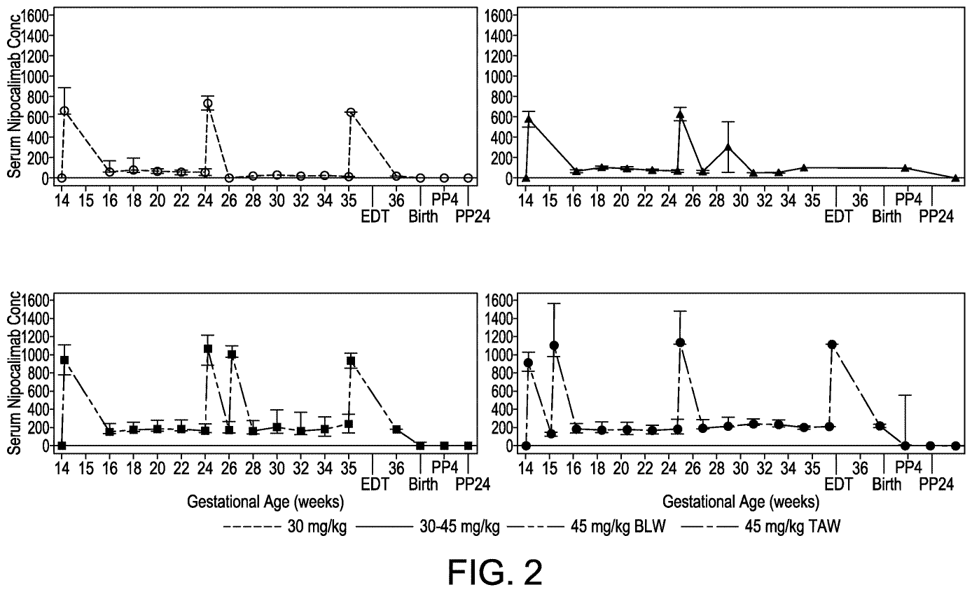

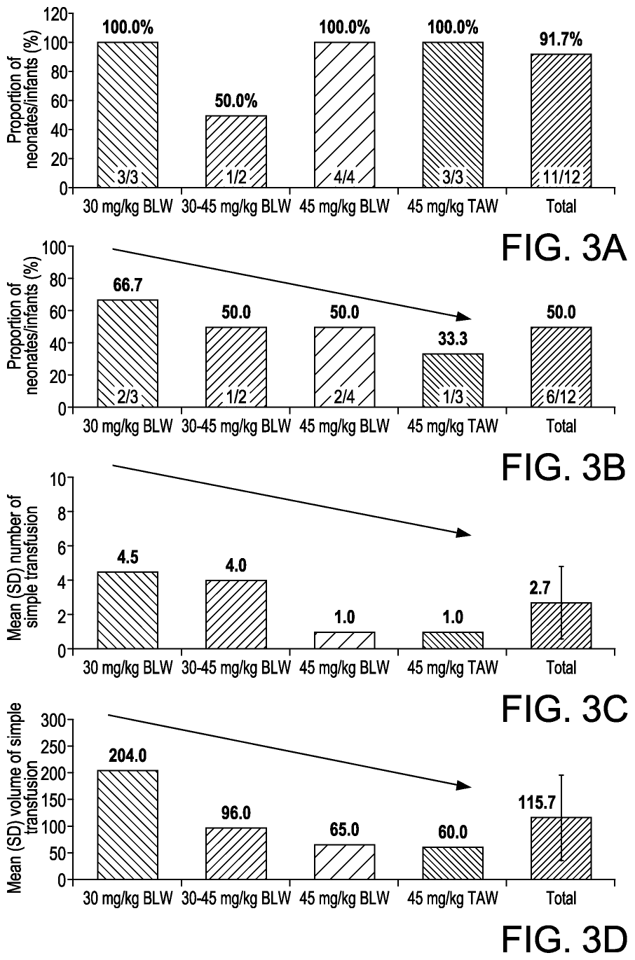

Further analysis comparing each regimen group were conducted. FIG. 1A shows the proportion of participants with live birth at or after GA Week 32 and without an IUT throughout entire pregnancies. As demonstrated herein, among, the different regimen groups, 30 mg/kg BLW regimen group had a lower rate (33.3%) of participants with live birth compared to other regimen groups. FIG. 1B shows that the 45 mg/kg dose group (BLW and TAW) has the highest proportion (62.5%) of maternal subjects with live birth at or after GA Week 32 and without an IUT throughout entire pregnancies. FIG. 2 shows median serum nipocalimab concentrations tended to be higher with decreased variability towards the end of the pregnancy (from GA Week 24 and on) with the 45 mg/kg BLW and 45 mg/kg TAW doses, indicating that less variable and more consistent exposures were achieved using the 45 mg/kg dose as compared to 30 mg/kg IV weekly BLW dosing. FIGS. 3A-3D show the neonatal outcomes among each regiment group, A) proportions of neonates requiring phototherapy; B) proportions of neonates and infants requiring simple transfusions; C) mean number of simple transfusions required by neonates and infants; and D) mean volume of simple transfusions required by neonates and infants. It can be seen here, the 30 mg/kg BLW group had the highest proportion of neonates and infants requiring simple transfusion, the highest mean number of simple transfusions, and the highest mean volume of simple transfusions, compared to other regimen groups.

### Safety:

SAEs were mainly related to HDFN or pregnancy and occurred with no discernable pattern or relationship to nipocalimab dose or pharmacodynamic effects. All 13 maternal participants had 1 or more treatment-emergent AEs (TEAEs); 12 (92.3%) maternal participants had a TEAE possibly, probably, or definitely related to nipocalimab. The most common TEAEs by preferred term, occurring in 4 or more maternal participants across all dose levels, were oedema peripheral (46.2%), nausea (30.8%), back pain (30.8%), and hypoalbuminaemia (30.8%). Study medication was permanently stopped due to a TEAE in 4 (30.8%) maternal participants. The number of maternal participants who had serious adverse events (SAEs) was 5 (38.5%); of those, 2 (15.4%) maternal participants had nipocalimab-related serious AEs (SAEs). 5 (38.5%) maternal participants experienced an AE of special interest (AESI). No maternal participants died during the study. One maternal participant experienced an AE of fetal loss. All 12 neonates/infants had 1 or more AEs; 7 (58.3%) neonates/infants had an AE possibly, probably, or definitely related to maternal nipocalimab exposure. The most common AEs by preferred term, occurring in 4 or more neonates/infants, were anaemia neonatal (58.3%), blood immunoglobulin G decreased (41.7%), hyperbilirubinaemia neonatal (41.7%), jaundice neonatal (33.3%). The number of neonates/infants who had SAEs was 5 (41.7%); of those, 2 (16.7%) neonates/infants had SAEs related to maternal nipocalimab exposure. 4 (33.3%) neonates/infants experienced an AESI. No neonates/infants died during the study.

### Pharmacokinetics:

Maternal serum nipocalimab concentrations were higher in the 45 mg/kg IV QW dose groups (BLW and TAW) compared to the 30 mg/kg IV QW BLW dose group. Furthermore, median serum nipocalimab concentrations tended to be higher with decreased variability towards the end of the pregnancy (from GA Week 24 and on) with the 45 mg/kg IV QW TAW dosing as compared to BLW dosing. Median maternal serum nipocalimab levels at birth were generally undetectable or close to undetectable except in one maternal participant who delivered the same day as receiving a dose in the morning. By postpartum Week 4, maternal serum levels of nipocalimab were undetectable across all dose groups. Neonatal serum nipocalimab concentrations at birth (Day 0) were detectable in 1 neonate (0.7 μg/mL) in the maternal 30 to 45 mg/kg BLW group only at birth, 8 days post the last maternal dose; in this neonate, considering absence of full RO when measured at birth (timepoint of detectable serum nipocalimab) and that IgG observations were comparable to that of other neonates, the detectable serum nipocalimab at birth in this neonate was considered not clinically meaningful. serum levels were undetectable in all other dose groups. By Week 4, neonatal serum levels of nipocalimab remained undetectable in all other maternal dose groups. Nipocalimab was undetectable in 3 participants' colostrum samples (n=7) and in 7 participants' breastmilk samples (n=9). In 5 of 6 samples with detectable nipocalimab in colostrum or breastmilk, nipocalimab levels were lower than the threshold required for PD effects (10 μg/mL); 1 maternal participant who delivered 1 day after nipocalimab administration had a colostrum level of 68.4 μg/mL, which was considered not clinically meaningful based on the breastfed neonate's nipocalimab serum PK level at Week 1 (<LLOQ) and normal IgG level for neonatal age at each timepoint measured. Overall, due to low participant numbers, the clinical impact of nipocalimab in colostrum and breastmilk cannot be determined with certainty.

### Pharmacodynamics:

Following the first dose of nipocalimab at the GA Week 14 visit, receptor occupancy (RO) decreased in all dose groups from 100% unoccupied to mean (SD) of 2.12 (4.103) percent unoccupied by GA Week 16 in all maternal participants, indicating rapid full receptor occupancy. Full receptor occupancy was maintained throughout the treatment period until approximately 1-3 weeks following the last nipocalimab dose when full receptor occupancy was rapidly lost (>60% mean unoccupied receptors at birth). At Day 0 (birth), neonates had mean (SD) of 80.70 (25.077) (range: 51.1-118.2) percent unoccupied FcRn receptors in monocytes by nipocalimab (in 7 neonates). Maternal alloantibody titer values (a semiquantitative measure) were decreased compared to baseline after initiation of dosing in all subjects at all nipocalimab doses. Maximum decreases in titers were observed to be between 8 to 32-fold lower than baseline. Decreased alloantibody titers were generally maintained at below baseline values and increased after the last dose. At postpartum Week 4 or 24, titers returned to near baseline or above. Serum IgG decreased in maternal participants following the initial nipocalimab dose from a mean (SD) of 9.45 (2.035) g/L to approximately −80% to −85% change from baseline at the subsequent visit weeks during the dosing period, starting at GA Week 16. Following the last dose of nipocalimab, maternal serum IgG recovery began returning towards baseline, with mean (SD) of −36 (4) percent change from baseline at birth (measured in 45 mg/kg BLW or TAW treated participants only). All maternal participants were observed with IgG values similar to baseline values by postpartum Week 4.

The persistence of high trough serum nipocalimab concentrations, full RO, total IgG, alloantibody lowering, and neonatal outcomes observed in UNITY support the testing of 45 mg/kg IV weeklyTAW dose in the Phase 3 Study.

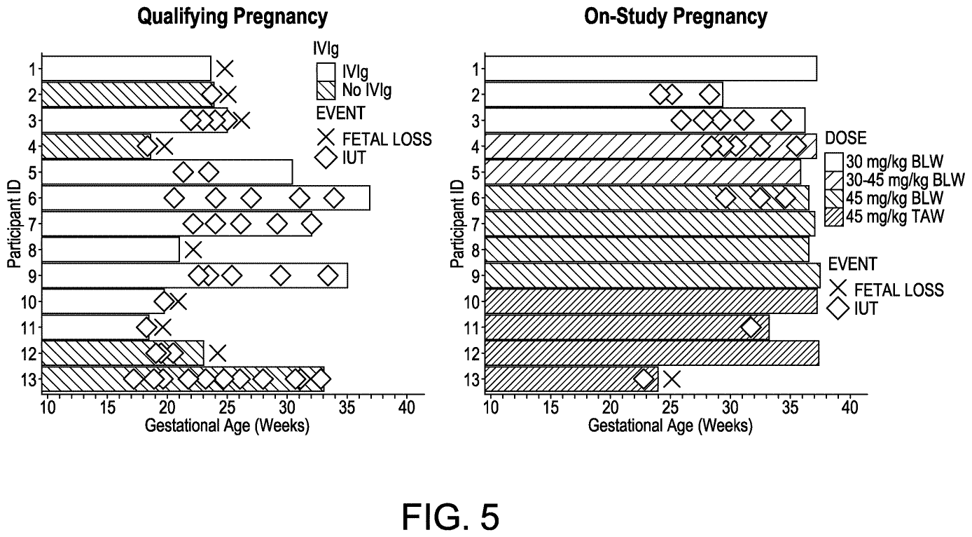

In addition, for each of the 13 participants, the participant's on-study pregnancy were compared with a previous qualifying pregnancy that met the criteria listed under item 4 of the inclusion criteria. As shown in FIG. 5, in contrast to on-study pregnancies, none of qualifying pregnancies had outcomes consistent with the primary endpoint (live birth at or after GA Week 32 without an IUT through the entire pregnancy). The proportion of participants with live births was 38% (5/13) in qualifying pregnancies and 92% (12/13) in on-study pregnancies. IUTs were required in 85% of qualifying pregnancies (IUTs per pregnancy range, 1-11) and 46% of on-study pregnancies (IUTs per pregnancy range, 1-5). In those receiving IUTs, median GA at first IUT was 20\hat{ }4/7 weeks in qualifying pregnancies and 27\hat{ }1/7 weeks in on-study pregnancies. Median (range) number of IUTs per pregnancy with live birth was 5 (2-11) in qualifying pregnancies and 3 (1-5) in on-study pregnancies. Fetal loss due to IUT complications was reported in 31% (4/13) of qualifying pregnancies and 8% (1/13) of on-study pregnancies. Fetal hydrops was observed in 54% (7/13) of qualifying pregnancies and no for on-study pregnancies. Median GA at delivery with a live birth was 23\hat{ }6/7 weeks in qualifying pregnancies and 36\hat{ }5/7 weeks in study pregnancies.

For postnatal outcomes, simple RBC transfusions were required in 80% (4/5) of neonates/infants from qualifying pregnancies and 50% (6/12) from on-study pregnancies. One of the 6 neonates/infants from on-study pregnancies who received simple RBC transfusions also received a single exchange transfusion and achieved the primary endpoint. Of the 7 on-study pregnancies that achieved the primary outcome, 6 neonates/infants did not require any antenatal/postnatal transfusions, indicating that 46% (6/13) of all on-study fetuses/neonates/infants did not undergo any invasive interventions.

Example 2. A prospective observational study designed to collect medical and treatment information on current treatment of EOS-HDFN in pregnant women and their offspring (CLARITY, ClinicalTrials.gov Identifier: NCT03755128). The purpose of the study was to provide data on the natural history of a similar population as studied in UNITY (Example 1). The CLARITY study was conducted in parallel to the UNITY study at overlapping clinical centers representing national and regional expert referral centers. CLARITY represents typical care at these expert referral centers for advanced monitoring and IUTs to treat severe HDFN. Approximately 50% of participants in CLARITY were administered IVIG (Intravenous immune globulin) in the treatment of EOS HDFN. This corresponds to treatment in the historic Benchmark. Of note, IVIG is not approved for the treatment of HDFN and limited data including small RCTs (Randomized Controlled Trial) or case series and is not included in the ACOG or SMFM treatment guidelines. Use of IVIG was not permitted during most of the UNITY study duration except just prior to birth of the fetus.

Total time on study was approximately 48 months for each pregnant woman and each child born during the study. This includes enrollment and pregnancy, a 6-month postnatal follow-up period for mothers, and a 1-year follow-up period for all neonates/infants.

Primary efficacy variable/Primary timepoint: At-risk pregnancies with a live birth at or after GA Week 32 and without an intrauterine transfusion (IUT) throughout their at-risk pregnancy.

### Selected Secondary Efficacy Variables:

At-risk pregnancies with live birth
Maternal participants with live birth without an IUT before GA Week 24
GA at first IUT and delivery
IUTs per maternal participant
Maternal participants with a fetus with fetal hydrops
Neonatal outcomes (neonates requiring phototherapy, simple transfusions in the first 3 months, and/or exchange transfusions)

15 pregnant women with similar key inclusion criteria as the UNITY study were enrolled in this study. In total, 10 of 15 (66.7%) maternal participants completed the study (i.e., did not discontinue early and completed the 6-month postpartum follow-up). Of the 8 participants that received IVIG, 2 achieved the primary outcomes while 6 participants did not achieve the primary outcome. Of the 7 participants that did not receive IVIG during their pregnancy, 0 achieved the primary outcome.

Based on the analysis of the primary efficacy variable, 13.3% (2/15) (95% CI: 1.7%, 40.5%) of maternal participants achieved the primary endpoint (live birth at or after GA Week 32 without an IUT through the entire pregnancy). This 13.3% rate is consistent with the historical benchmark of 10%, and much lower than 53.8% rate achieved in UNITY study wherein nipocalimab was administered.

Based on the Maternal Subjects (N=15) analysis:
14 (93.3%) of maternal participants had a live birth at any time (95% CI: 68.1%, 99.8%).
9 (60.0%) of maternal participants had a live birth, without an IUT before GA Week 24 (95% CI: 32.3%, 83.7%). Of those 9, 7 (46.7%) maternal participants had an IUT after GA Week 24.
Mean GA at any delivery outcome (live birth or fetal loss), with or without IUT, is Week 34\hat{ }2/7 (range: Week 16\hat{ }5/7 to Week 38\hat{ }0/7), with 7 (46.7%) maternal participants having delivery outcome at GA ≥37 weeks.
For the 12 (80.0%) maternal participants with live births who had an IUT, the mean GA at first IUT is Week 22\hat{ }5/7 (range: Week 17\hat{ }1/7 to Week 28\hat{ }2/7).
1 (6.7%) maternal participants had a fetus with fetal hydrops.

Based on the Neonates/Infants (N=14) analysis:
14 (100%) neonates/infants had at least 1 phototherapy session.
8 (57.1%) neonates/infants had at least 1 simple transfusion in the first 3 months.
3 (21.4%) neonate/infant had an exchange transfusion.

### Safety:

2 (13.3%) maternal participants had 1 or more serious AE (SAE). These SAEs, by system organ class (SOC) and preferred term, were pregnancy, puerperium and perinatal conditions (abortion spontaneous) and skin and subcutaneous tissue disorders (scar pain). No maternal participants experienced an AE of special interest (AESI).
No maternal participants died during the study; however, one maternal participant experienced a fetal loss.
1 (7.1%) neonate/infant had 1 or more SAE. This SAE, by SOC and preferred term, was investigations (blood bilirubin increased). 1 (7.1%) neonate/infant experienced an AESI.
No neonates/infants died during the study.

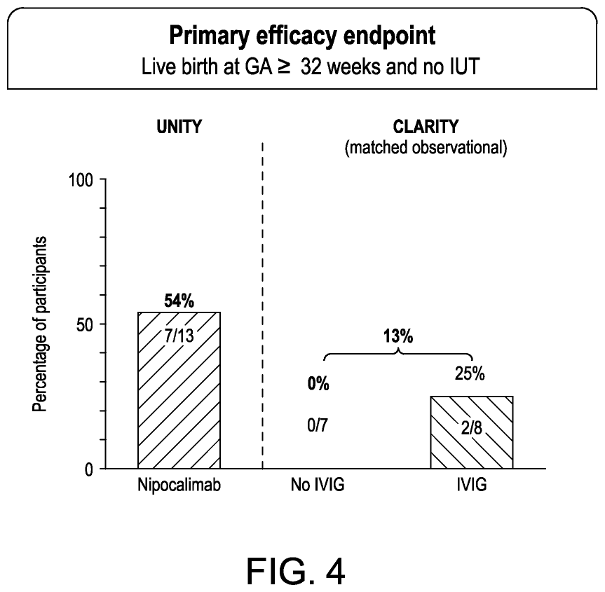

As shown in FIG. 4, 54% of participants (7/13) met the primary efficacy endpoint (live birth at or after GA Week 32 without an IUT through the entire pregnancy) in UNITY study, while only 13% of participants met the primary efficacy endpoint in CLARITY study. And in CLARITY study, no participants (0/7) met the primary efficacy within the group not using IVIG.

Example 3. Phase 3 Study Design. This is a Phase 3 randomized, placebo-controlled, double-blind, multicenter study to evaluate the efficacy and safety of nipocalimab in pregnancies at risk for severe HDFN. Summary of objectives (primary and secondary) and endpoints is shown in Table 1 below.

TABLE 1

| | |
|---|---|
| Objectives | Endpoints |

Primary

| | |
|---|---|
| To evaluate the | Primary endpoint: |
| efficacy of | The proportion of pregnancies that did not result in fetal loss |
| nipocalimab | (due to any reason), intrauterine transfusion (IUT), hydrops |
| compared with | fetalis, or neonatal death (due to any reason) during the neonatal |
| placebo on reducing | period (through 4 weeks of age or 41 weeks PMA, whichever is |
| the risk of fetal | later). |
| anemia with live | |
| neonates in pregnant | |
| women at risk for | |
| severe HDFN | |

Secondary

| | |
|---|---|
| To evaluate the | Key secondary endpoint: |
| efficacy of | The severity of HDFN. For the analysis purpose, the severity of |
| nipocalimab | HDFN is defined as: |
| compared with | 5 (Fatal): fetal or neonatal death due to any reason |
| placebo in reducing | 4 (Severe): hydrops fetalis (in fetus or newborn) or |
| the severity of HDFN | receiving IUT during pregnancy as a result of HDFN but |
| as measured by a | not 5 (Fatal) |
| composite severity | 3 (Moderate): neonatal exchange transfusions received as |
| index in pregnant | a result of HDFN related hemolysis and jaundice but not 4 |
| women and their | (Severe) or 5 (Fatal) |
| neonates/infants at | 2 (Mild): neonatal simple transfusions received due to |
| risk for severe HDFN | HDFN after birth, with or without phototherapy, but not |
| | 3 (Moderate), 4 (Severe), or 5 (Fatal) |
| | 1 (Minimal or None): not in 2 (Mild), 3 (Moderate), 4 |
| | (Severe), or 5 (Fatal) as described above. HDFN requiring |
| | phototherapy is classified in this category |
| | Note: At the first DBL (4 weeks after birth), data through the Week |
| | 4 visit or 41 weeks PMA (whichever is later) is used to calculate the |
| | HDFN severity index. At the second DBL (24 weeks after birth), |
| | data through 12 weeks after birth is used to calculate the HDFN |
| | severity index. |
| To evaluate the | Key Secondary Endpoint: |
| efficacy of | The earliest time of IUT or hydrops fetalis |
| nipocalimab | |
| compared with | |
| placebo on delaying | |
| the onset of severe | |
| HDFN in pregnant | |
| women at risk for | |
| severe HDFN | |
| To evaluate the | Key Secondary endpoint: |
| impact of | The number of IUTs received during pregnancy |
| nipocalimab | Other endpoints: |
| compared with | The proportion of pregnancies with fetal loss |
| placebo on antenatal | The proportion of pregnancies with fetal or neonatal death |
| HDFN management | (through the neonatal period) as a result of HDFN |
| and outcomes in | The proportion of pregnancies with hydrops fetalis |
| pregnant women at | The proportion of pregnancies receiving IUT during pregnancy. |
| risk for severe | Gestational age at first IUT |
| HDFN | The proportion of pregnancies receiving >1 IUT during |
| | pregnancy |
| | The proportion of pregnancies receiving IUT or HDFN |
| | resulting in fetal demise <GA Week 20\hat{ }0/7 |
| | Gestational age at delivery |
| To evaluate the | Key Secondary Endpoint: |
| efficacy of | The modified neonatal mortality and morbidity index in |
| nipocalimab | liveborn neonates through 38 weeks PMA or at discharge if |
| compared with | earlier than 38 weeks PMA |
| placebo on reducing | |
| the risk of mortality | |
| and morbidity in | |
| neonates born to | |
| women at risk for | |
| severe HDFN | |
| To evaluate the | Other endpoints: |
| impact of | The proportion of pregnancies with neonatal death through the |
| nipocalimab | neonatal period |
| compared with | The proportion of liveborn neonates with HDFN-related |
| placebo on neonatal | morbidities other than anemia and hyperbilirubinemia/jaundice |
| HDFN management | Absolute weight and weight changes over time from birth |
| and other outcomes | through Week 104 in liveborn neonates/infants |
| of HDFN in | Liveborn neonates length of stay in neonatal intensive care unit |
| neonates/infants of | The proportion of liveborn neonates receiving exchange |
| pregnant women at | transfusions for HDFN |
| risk for severe | Number of neonatal exchange transfusions per liveborn neonate |
| HDFN | The proportion of liveborn neonates/infants with simple |
| | transfusions for HDFN through the neonatal period (for the first |
| | DBL) or 12 weeks (for the second DBL) after birth |
| | Number of simple transfusions for HDFN per liveborn |
| | neonate/infant through the neonatal period (for the first DBL) |
| | or 12 weeks (for the second DBL) after birth |
| | The proportion of liveborn neonates with hyperbilirubinemia |
| | treated with phototherapy |
| | Number of days of phototherapy received for |
| | hyperbilirubinemia per liveborn neonate |
| | The proportion of liveborn neonates/infants receiving IVIg for |
| | HDFN treatment |
| To evaluate the | Other endpoints: |
| safety of | Maternal/fetal safety outcomes: |
| nipocalimab in | Maternal death, adverse events (AEs), serious adverse events |
| pregnant women at | (SAEs), AEs of special interest (AESIs), AEs leading to |
| risk for severe | discontinuations, infections, serious infections, infusion |
| HDFN, including | reactions, hypersensitivity reactions |
| pregnancy outcomes | Maternal pregnancy complications |
| compared with | IUT related complications |
| placebo | Pregnancy outcomes: |
| | The proportion of pregnancies with: |
| | Cesarean delivery |
| | Cesarean delivery due to IUT complications |
| | Preterm birth <GA Week 28\hat{ }0/7 |
| | Preterm birth <GA Week 32\hat{ }0/7 |
| | Preterm birth <GA Week 34\hat{ }0/7 |
| | Preterm birth <GA Week 37\hat{ }0/7 |
| | Fetal growth restriction |
| | Preeclampsia |
| To evaluate the | Other endpoints: |
| safety of | Neonates/infants safety and development outcomes: |
| neonates/infants born | The proportion of liveborn neonates/infants who died |
| to nipocalimab- | The proportion of liveborn neonates/infants with AEs, SAEs, |
| treated mothers | AESIs, infections, serious infections |
| compared with | The proportion of liveborn neonates/infants receiving IVIg for |
| neonates/infants born | non-HDFN indications |
| to mothers who | The proportion of liveborn neonates/infants with abnormal |
| received placebo | hearing |
| | Bayley Scales at Week 52 and Week 104 in infants |
| To evaluate the | Other endpoints: |
| impact of | Maternal Patient-reported Outcomes (PROs): |
| nipocalimab on | Change from baseline in Generalized Anxiety Disorder 7-item |
| patient- and | (GAD-7) over time during pregnancy and postpartum |
| caregiver-reported | Change from baseline in domain scores and physical component |
| outcomes in | summary and mental component summary in 36-Item Short |
| participants at risk | Form Health Survey version 2 Acute (SF-36 v2 Acute) over |
| for severe HDFN and | time during pregnancy and postpartum |
| their neonates/ | Change from baseline in EuroQol five-dimension questionnaire |
| infants | (EQ-5D-5L) Visual Analogue Scale (VAS) and in EQ-5D index |
| | scores by visit over time during pregnancy and postpartum |
| | Neonate/infant caregiver-reported outcomes: |
| | Summary of IQI score over time |

Exploratory

| | |
|---|---|
| To evaluate the | Maternal/fetal endpoints: |
| efficacy of | Middle cerebral artery (MCA)- peak systolic velocity (PSV) |
| nipocalimab | multiples of the median (MoM) over time |
| compared with | Maternal alloantibodies over time during pregnancy and |
| placebo on other | postpartum |
| HDFN-related | Fetal hemoglobin, hematocrit, direct Coombs, and alloantibody |
| measurements | titers at first IUT and in subsequent IUTs |
| | Difference in GA at onset of fetal anemia between the current |
| | and most recent qualifying pregnancy |
| | Neonatal/infants' endpoints |
| | Direct Coombs and alloantibodies titers in neonates/infants over |
| | time |
| | Neonatal bilirubin, reticulocyte count, hemoglobin, and |
| | hematocrit at birth and over time |
| To evaluate the PK, | Serum nipocalimab concentration in maternal blood over time |
| PD, and | during pregnancy and postpartum |
| immunogenicity of | Incidence and titers of antibodies to nipocalimab and |
| nipocalimab in | neutralizing antibodies (Nabs) in maternal blood during |
| pregnant women at | pregnancy and postpartum |
| risk for severe | Total IgG in maternal blood over time during pregnancy and |
| HDFN | postpartum |
| To evaluate the PK | Nipocalimab concentration in blood collected at first IUT and |
| of nipocalimab in the | in subsequent IUTs, in cord blood at birth, and in blood of |
| fetus, neonate/infant, | infants |
| and in colostrum, | Nipocalimab concentration in colostrum, transitional milk, |
| transitional milk, | and/or mature breast milk |
| and/or mature breast | |
| milk | |
| To evaluate FcRn | FcRn RO in maternal blood over time during pregnancy and |
| receptor occupancy | postpartum |
| (RO) of nipocalimab | |
| in pregnant women | |
| at risk for severe | |
| HDFN | |
| To evaluate the PD | FcRn RO in blood collected at first IUT and in subsequent IUTs, |
| of nipocalimab in the | in cord blood at birth, and in blood of infants |
| fetus and | Total IgG and respective alloantibodies in blood collected at |
| neonate/infant | first IUT and in subsequent IUTs, in cord blood at birth, and in |
| | blood of infants over time |
| To evaluate the | Including but not limited to |
| effect of nipocalimab | IgA, IgM, IgE, and IgG subclasses in mothers and infants over |
| on biomarkers of | time |
| interest | Response to tetanus vaccine in mothers and infants |
| | Lymphocyte immunophenotyping |
| | Placental pathology evaluation |

The total duration of the study is up to 135 weeks and consists of 4 study periods: approximately <9 week screening period, a double-blind treatment period of up to 23 weeks, and safety follow up periods of 24 weeks following delivery for the mother and 104 weeks from birth for the neonate/infant. The timing of delivery is dependent on investigator judgment and is anticipated at GA Week 37 or 38, which is standard practice for pregnancies with severe HDFN.

Screening procedures are completed between GA Week 8\hat{ }0/7 (i.e., 8 weeks, the earliest time for a maternal participant to consent) and prior to randomization (Day 1/Baseline). The randomization period occurs from GA Week 13\hat{ }0/7 (ie, 13 weeks) to GA Week 16\hat{ }3/7 (ie, 16 weeks and 3 days). Day 1 dosing should occur on days 0/7 to 3/7 of the GA Week during the randomization period (ie, GA Weeks 13 to 16). On Day 1, an ultrasound is performed to confirm no signs of fetal anemia or hydrops and MCA-PSV <1.5 MoM before randomization. The timing of screening procedures must take into account the turnaround time for availability of laboratory tests needed for eligibility assessment, including testing for cffDNA. The optimal timing for collection of the cffDNA sample is ≥10 Week GA and turnaround time for availability of results is up to 2 weeks from date of sample collection. Study intervention administration occurs on Day 1 after eligibility is confirmed. Weight measured on Day 1 is used to calculate the study intervention dose. At subsequent visits, weight measured at the prior week is used to calculate the study intervention dose.

Up to approximately 120 eligible pregnant women are randomized in a 2:1 (nipocalimab to placebo) ratio to receive nipocalimab 45 mg/kg weekly or matching placebo administered intravenously from randomization through GA Week 35. Permuted block randomization are used. The target study population is pregnant women with an obstetrical history of HDFN-related fetal anemia with IUT, hydrops fetalis, fetal loss, or neonatal death in a previous pregnancy and are currently pregnant with an antigen-positive fetus who are not receiving and have no plans to receive IVIg/plasmapheresis.

In both treatment groups, the participants are monitored weekly by MCA Doppler and IUT are performed when the PSV of blood flow in MCA is ≥1.5 MoM for GA or hydrops and fetal blood sampling confirms anemia.

In the postnatal follow-up phase, monitoring for neonates/infants includes hyperbilirubinemia, anemia, and IgG levels. While there are no universally accepted international guidelines for management of anemia and hyperbilirubinemia in neonates/infants, guidance for management for this study is provided.

Efficacy, safety, PK, immunogenicity, PD (e.g., FcRn RO and IgG), and biomarkers are assessed according to schedule.

Efficacy assessments include weekly MCA Doppler to assess fetal anemia and IUT received during the antenatal period, phototherapy or exchange transfusion and/or simple transfusion received to manage anemia and hyperbilirubinemia in the neonate/infant, the modified neonatal mortality and morbidity index, and other neonatal outcomes. In addition, fetal/neonatal hemoglobin, hematocrit, total bilirubin are assessed. Patient reported outcomes for the mother include GAD-7, SF-36v2 Acute, and EQ-5D-5L. Health related quality of life outcomes reported by caregivers for the neonate/infant are assessed by the IQI.

Safety assessments include AEs, infusion reactions, concomitant medications, clinical labs (hematology, chemistry [including lipid panel and urinalysis), ECG, vital signs, maternal and neonatal/infant body weight, physical exams, ultrasound monitoring of fetal growth and development, hearing screening for the neonate, Bayley Scales for infant development (3rd edition), and neonatal/infant immune development (Ig profile, lymphocyte phenotyping, vaccine response to tetanus). Local and central pathology review of placentas are performed.

Study Population: The target study population is pregnant women with an obstetrical history of HDFN-related fetal anemia with IUT, hydrops fetalis, fetal loss, or neonatal death in a previous pregnancy and are currently pregnant with an antigen-positive fetus who are not receiving and have no plans to receive IVIg/plasmapheresis. These pregnancies are at risk for severe HDFN with fetal anemia during the current pregnancy, and are considered appropriate to provide relevant efficacy and safety information of nipocalimab with a randomized, double-blind, placebo-controlled design for the intended use of nipocalimab if approved for severe HDFN. Maternal alloimmunity to the RBC antigens most commonly implicated in severe HDFN (RhD, Kell, Rhc, RhE, and RhC) allows for the examination of efficacy across different RBC antigens.

Blinding, Control, Study Phase/Periods, Intervention Groups: A placebo control are used to establish the frequency and magnitude of changes in clinical endpoints that may occur in the absence of active intervention. A placebo control is considered appropriate for this target population, since all participants have standard of care available to them. Participants are closely monitored for fetal anemia via weekly assessment of the MCA-PSV by Doppler ultrasonography and receive IUT when fetal anemia is detected.

The nipocalimab dose regimen selected for evaluation in this study in pregnant women at risk for severe HDFN is as follows: IV administration of 45 mg/kg of nipocalimab given qw initiated between GA Week 13\hat{ }0/7 and GA Week 16\hat{ }3/7 continuing through GA Week 35 (i.e., approximately 2 3 weeks prior to the planned delivery at GA Week 37 or 38), based on weight measurements throughout the pregnancy to account for weight increase during pregnancy.

This dose regimen is selected based on the PK, PD, efficacy, and safety data from completed and ongoing clinical studies of nipocalimab, including the following: 1) Phase 1 FIH SAD/MAD study in healthy participants (MoM-M281-001) where single doses of nipocalimab up to 60 mg/kg and multiple doses up to 30 mg/kg weekly were evaluated, 2) a Phase 2 study in gMG (MoM-M281-004) where doses up to 60 mg/kg every 2 weeks for 8 weeks were evaluated, and 3) the available data from the ongoing open-label study in HDFN (MoM-M281-003) with 30 mg/kg or 45 mg/kg weekly dosing (using the weight measured at baseline and time-adjusted weight-based dosing). Also contributing to the dose selection for this Phase 3 HDFN study were the simulations based on the PK/PD (including FcRn Receptor Occupancy and IgG) model using the FIH data.

Participants are randomized in a 2:1 (nipocalimab to placebo) ratio to 1 of 2 treatment groups, respectively, as described below:
Group 1: nipocalimab 45 mg/kg IV QW: Participants receive nipocalimab 45 mg/kg IV QW through GA Week 35
Group 2: placebo IV QW: Participants receive placebo IV QW through GA Week 35

Study intervention is administered weekly by IV infusion at a dose of 45 mg/kg based on the participant's weight (rounded to the nearest 0.1 kg) beginning from randomization Day 1 to GA Week 35. Weight measurement is used to calculate the study intervention dose as follows:
On Day 1, the weight measured at the Day 1 visit is used to calculate the dose of study intervention to be administered at that visit.
For subsequent visits, the weight measured at the prior week is used to calculate the study intervention dose to be administered. If the weight measurement from the prior week is missing or if the last dose is ≥9 days, the weight measured on the same day as the study intervention infusion is used. Adjusting the dose of the composition according to the weight of the participant as measured at the prior week is intended to provide a more efficacious treatment and to improve patient outcomes, compared to embodiments in which the dose of the composition is based only on the weight of the participant at the start of the study. Basing the dose of the composition based on the weight of the participant at the prior week of administration also allows the composition be prepared prior to the day of administration.

The maximum dose amount given to any participant at any dosing visit does not exceed 5.4 grams, regardless of the participant's body weight. Day 1 (first dose of study intervention) occurs on days 0/7 to 3/7 of the GA week during the randomization period (ie, GA Weeks 13 to 16). All weekly study intervention infusion after Day 1 occurs every 7 days (±1 day) counting from Day 1. “QW” stands for weekly. The minimum interval between 2 doses is 6 days, and the maximum interval between 2 doses does not exceed 8 days. Each participant receives study intervention a minimum of 20 weeks (GA Week 16 to GA Week 35) and a maximum of 23 weeks (GA Week 13 to GA Week 35) if not meeting discontinuation criteria. In the event of unscheduled (e.g., urgent or emergency) delivery, study intervention administration are stopped as soon as the decision is made to deliver early. If a dose is delayed or interrupted (e.g., ≥9 days from the most recent dose) subsequent weekly study drug administration and study visit occurs every 7 days (±1 day) counting from the date the study intervention administration resumed. Study intervention administration, including start and stop dates and times (including for delayed and/or interrupted events) are captured in the source documents and the eCRF.

The following inclusion and exclusion criteria are followed when screening for participants:

Inclusion criteria include:
1. Female, 18 (or the legal age of consent if above 18 in local regions) to 45 years of age, inclusive, at the time of informed consent.
2. Pregnant and an estimated GA (based on ultrasound dating) between Week 13\hat{ }0/7 and Week 16\hat{ }3/7 (i.e., 13 weeks to 16 weeks and 3 days) at randomization.
3. History of severe HDFN in a prior pregnancy defined as: a) documented fetal anemia (defined as fetal hemoglobin level <0.84 MoM. Fetal hemoglobin (g/dL) can be calculated using hematocrit (%)/3), or received ≥1 IUT as a result of HDFN; or b) fetal loss or neonatal death as a result of HDFN, with maternal alloantibody titers for RhD, Kell, Rhc, RhE, or RhC antigen above the critical levels (anti Kell ≥4; other ≥16) and evidence of an antigen-positive fetus.
4. During the current pregnancy, presence of maternal alloantibody to RhD, Kell, Rhc, RhE, or RhC antigen with titers above the critical level (anti-Kell ≥4; other ≥16), based on the designated central lab results at screening.
5. Evidence of an antigen-positive (RhD, Kell, Rhc, RhE, or RhC) fetus corresponding to the historical maternal alloantibody confirmed by non-invasive cffDNA performed at the central laboratory.
6. Have screening laboratory test results within the following parameters based on the central laboratory results: a. Albumin ≥2.6 g/dL (International System of Units [SI]: >26 g/L); b. Aspartate aminotransferase (AST)≤2×upper limit of normal (ULN)*; c. Alanine aminotransferase (ALT)≤2×ULN*; d. Creatinine≤0.8 mg/dL (SI: ≤70.7 μmol/L); e. serum total IgG ≥600 mg/dL (SIL ≥6 g/L); *Note: values for ULN are based on normal reference range for the appropriate gestational age of pregnancy (Abbassi-Ghanavati M (2009), Greer L G, Cunningham F G. Pregnancy and laboratory studies: a reference table for clinicians [published correction appears in Obstet Gynecol. 2010 February; 115 (2 Pt 1): 387]. Obstet Gynecol. 2009; 114 (6): 1326-1331.).
7. Otherwise healthy on the basis of physical examination, medical history, vital signs, 12-lead ECG, and clinical laboratory tests performed at screening.
8. Willing to receive standard of care treatment with IUT, if clinically indicated.
9. Agrees to receive recommended vaccinations according to local standard of care for both mother and infant throughout the course of the study.
10. Willing to forego collection of cord blood for stem cell storage or other non-study purposes.
11. For mother and neonate, willing to forego participation in another clinical study of an investigational therapy for the duration of their participation in the current study.
12. Must sign an ICF indicating that she understands the purpose of, and procedures required for, the study and is willing to participate in the study and consents to a 24-week safety follow-up period. An additional consent may be obtained if needed according to local requirements. The parents/guardian of the neonates/infant must also sign an ICF to permit 104-week follow up for the neonate/infant and agree to complete caregiver-reported outcomes for the infant.
14. Must be able to read and write.
15. Must agree not to donate blood through the final follow-up visit at Week 24 postpartum.

### Exclusion Criteria Include:

1. Currently pregnant with a multiple gestation (twins or more);
2. Evidence of fetal anemia by ultrasound or repeated MCA-PSV >1.5 MoM prior to randomization in the current pregnancy;
3. History of severe early-onset preeclampsia prior to GA Week 34 or severe FGR (EFW <3^{rd} percentile) in a previous pregnancy;
4. Current unstable hypertension;
5. History of myocardial infarction, unstable ischemic heart disease, or stroke;
6. Has a history of severe and/or uncontrolledhepatic (e.g., viral/alcoholic/autoimmune hepatitis/cirrhosis, and/or metabolic liver disease), gastrointestinal, renal, pulmonary, cardiovascular, psychiatric, neurological, musculoskeletal disorder, hypertension, and/or any other medical or uncontrolled autoimmune disorder(s) (e.g., diabetes mellitus), that might interfere with the participant's full participation in the study, and/or might jeopardize the safety of the participant; (Note: Any condition (e.g., COVID-19) for which, in the opinion of the investigator, participation would not be in the best interest of the participant (e.g., compromise well-being) or that could prevent, limit or confound the protocol-specified assessments.)
7. Has any confirmed or suspected clinical immunodeficiency syndrome or has a family history of congenital or hereditary immunodeficiency unless confirmed absent in the participant;
8. History of solid organ or bone marrow transplantation (with the exception of a corneal transplant performed >12 weeks before screening);
9. Has inflammatory or autoimmune diseases requiring immunosuppressive therapies that may jeopardize the safety of the participant;
10. Currently has a malignancy or has a history of malignancy within 3 years before screening (with the exception of localized basal cell carcinoma and/or squamous cell carcinoma skin cancer that has been adequately treated with no evidence of recurrence for at least 3 months [defined as a minimum of 12 weeks] before the first study intervention administration or cervical carcinoma in situ that has been treated with no evidence of recurrence for at least 3 months before the first study intervention administration);
11. Known allergies, hypersensitivity, or intolerance to excipients in the study intervention;
12. Has shown a previous severe immediate hypersensitivity reaction such as anaphylaxis to therapeutic proteins (e.g., mAbs);
13. Has previously received Nipocalimab, or was enrolled and received study intervention in this study in a previous pregnancy;
14. Has previously received batoclimab, efgartigimod, rozanolixizumab, or other anti-FcRn investigational agents;
15. Is currently receiving systemic corticosteroids or other immunosuppressants for disorders unrelated to the pregnancy (Note: use of low-potency topical corticosteroids, nasal/inhaled corticosteroids, or intra-articular corticosteroids is permitted. Use of systemic corticosteroids is permitted prior to delivery for lung maturation as needed);
16. Has received or planning to receive plasmapheresis, immunoadsorption therapy, IVIg, or any IgG Fc-related protein therapeutics during the current pregnancy;
17. Has previously received rituximab or eculizumab within 6 months prior to screening;
18. Has received a live virus vaccination during the current pregnancy or has a known need to receive a live virus vaccination during the study while receiving study intervention or within at least 8 weeks after the last administration of study intervention in this study;
19. Has received an investigational intervention (including investigational vaccines except emergency use-authorized COVID-19 vaccines) within 3 months or 5 half-lives (whichever is longer) prior to first administration of study intervention or is currently enrolled or plan to enroll in an investigational study;
20. Has a severe infection including opportunistic infections (e.g., pneumonia, biliary tract infection, diverticulitis, Clostridium difficile infection, cytomegalovirus, pneumocystosis, aspergillosis, etc.) requiring parenteral anti-infectives and/or hospitalization, and/or is assessed as serious/clinically significant by the investigator, during the current pregnancy;
21. Has a history of active granulomatous infection, including histoplasmosis or coccidioidomycosis, before screening;
22. Active infection during screening or on Day 1 (prior to randomization of participants) with Coxsackie, syphilis, cytomegalovirus, toxoplasmosis, or herpes simplex 1 or 2, as evidenced by clinical signs and symptoms and serology results from the central laboratory (Note: Patients with serologic evidence of prior infection or exposure but without clinical signs and symptoms of active infection can be considered for participation);
23. History of severe or recurrent pyelonephritis or 4 or more lower urinary tract infections in the past year or in a previous pregnancy;
24. Has a chronic infection (e.g., bronchiectasis, chronic osteomyelitis) or requires chronic treatment with anti-infectives (e.g., antibiotics, antivirals);
25. History of atypical mycobacterial disease or herpes zoster infection within the last 6 months;
26. Tests positive for hepatitis B virus (HBV) infection;
27. Is seropositive for antibodies to hepatitis C virus (HCV), unless they satisfy 1 of the following conditions: a) Has a history of successful treatment, defined as being negative for HCV ribonucleic acid (RNA) at least 24 weeks after completing antiviral treatment, and has a negative HCV RNA test result at screening, OR b) While seropositive, has a negative HCV RNA test result at least 24 weeks prior to screening and a negative HCV RNA test at the screening.
28. History of being human immunodeficiency virus (HIV) 1 or HIV2 antibody-positive, or tests positive for HIV at screening;
29. COVID-19 infection (Have tested positive for or been exposed to COVID-19 within 4 weeks prior to the first dose of study intervention; Exceptions: Participants who have tested positive for or been exposed to COVID-19 may participate if they have both an absence of symptoms and a negative validated COVID-19 test obtained at least 2 weeks after symptom onset (or the first positive test for asymptomatic infection) or exposure; Follow local regulations/guidelines for validated COVID-19 testing procedures and standard definition of COVID-19 exposure);
30. History of drug or alcohol abuse according to Diagnostic and Statistical Manual of Mental Disorders (5th edition) (DSM-V) criteria within 1 year before screening;
31. Has any condition (including known genetic defects of the fetus or umbilical cord abnormality) for which, in the opinion of the investigator, participation would not be in the best interest of the participant or fetus/neonate/infant (e.g., compromise the well-being) or that could prevent, limit, or confound the protocol-specified assessments.
32. Has had a BCG vaccination within 1 year prior to the first administration of study intervention or has a known need to receive a BCG vaccine during the study or within at least 8 weeks after the last administration of study intervention;
33. Presence of abnormal hematologic laboratory values during screening:

| | | | |
|---|---|---|---|
| a. | Hemoglobin | <8.0 g/dL | SI: <80 g/L |
| b. | WBC | <3.0 × 103/μL | SI: <3.0 GI/L |
| c. | Neutrophils | <1.5 × 103/μL | SI: <1.5 GI/L |
| d. | Platelets | <100 × 103/μL | SI: <100 GI/L |

Note:
(The inclusion of any participant with hemoglobin levels between 8.0 and 9.0 g/dL must be discussed with the sponsor to decide whether the participant can be included in the study).

Example 4. Treatment of hemolytic disease of the fetus and newborn with nipocalimab does not elevate cholesterol. Elevations in total cholesterol and low-density lipoprotein (LDL) were reported recently with another experimental anti-FcRn antibody that is not nipocalimab in the same pharmacological class of FcRn antagonists. This finding triggered a review of lipid data in Sponsor-completed and ongoing nipocalimab studies. In the Phase 1 healthy volunteer MoM-M281-001 and Phase 2 generalized myasthenia gravis MoM-M281-004 studies, asymptomatic, dose dependent, reversible elevations in non-fasting mean total cholesterol were observed up to 25% of baseline. In study MoM-M281-004, at the highest dose of 60 mg/kg every 2 weeks (Q2W), the mean percent change in total cholesterol increased to a stable maximum of 21% to 23% above baseline within 1 month of initiation of dosing and declined to near baseline level 1-2 months after the last dose. As a result of these findings, the following assessments are conducted: 1) assessment for lipids (total cholesterol, HDL, calculated LDL, and triglycerides) in fasting and non-fasting conditions at multiple time points on and off treatment, 2) exclusion criterion for patients with a recent significant cardiovascular event, 3) recommendation for lipid abnormalities management according to local health guidelines.

This list of therapies is not all inclusive, it is recommended to consult with the sponsor prior to starting any biologic, non-biologic, or other advanced therapies.

The embodiments and examples provided herein demonstrate that an anti-FcRn antibody, such as, but not limited to, nipocalimab is effective to treat hemolytic disease of the fetus and newborn as measured one or more of the indices and outcomes as provided for herein.

The disclosures of each and every patent, patent application, and publication cited herein are hereby incorporated herein by reference in their entirety. While various embodiments have been disclosed with reference to specific aspects, it is apparent that other aspects and variations of these embodiments may be devised by others skilled in the art without departing from the true spirit and scope of the embodiments. The appended claims are intended to be construed to include all such aspects and equivalent variations.

1. A method of treating, or reducing severity of, hemolytic disease of the fetus and newborn (HDFN) in a fetal subject, the method comprising administering a composition comprising an anti-FcRn antibody to a pregnant woman carrying the fetal subject, wherein the anti-FcRn antibody comprises:
(a) a light chain, wherein the CDR L1 comprises the sequence of SEQ ID NO: 3, the CDR L2 comprises the sequence of SEQ ID NO: 4, the CDR L3 comprises the sequence of SEQ ID NO: 5; and
(b) a heavy chain, wherein the CDR H1 comprises the sequence of SEQ ID NO: 6, the CDR H2 comprises the sequence of SEQ ID NO: 7, and the CDR H3 comprises the sequence of SEQ ID NO: 8.

2. The method of claim 1, wherein the fetal subject is antigen positive.

3. The method of claim 1, wherein the pregnant woman is antigen negative, and wherein the antigen is RhD, Kell, Rhc, RhE, RhC, or any combination thereof.

4.-6. (canceled)

7. The method of claim 1, wherein the pregnant woman has maternal alloantibodies to the antigen, and wherein the maternal alloantibodies have a titer selected from:
greater than or equal to 4 for anti-Kell;
greater than or equal to 16 for anti-RhD;
greater than or equal to 16 for anti-Rhc;
greater than or equal to 16 for anti-RhE;
greater than or equal to 16 for anti-RhC; or
any combination thereof.

8. The method of claim 1, wherein the fetal subject has a reduction in maternal alloantibodies or total immunoglobulin (IgG) following administration of the composition.

9. The method of claim 1, wherein the pregnant woman has obstetrical history comprising a previous pregnancy with a previous fetus, wherein:
i) the pregnant woman received one or more intrauterine transfusions (IUTs) to treat fetal anemia during the previous pregnancy;
ii) the previous fetus suffered from fetal anemia; and/or
iii) the previous pregnancy resulted in fetal demise and the pregnant woman had detectable maternal alloantibody titers to one or more of RhD, Kell, Rhc, RhE, and RhC antigens above a critical level during the previous pregnancy, wherein said critical level of anti-Kell maternal alloantibody titer is ≥4, said critical level of anti-RhD maternal alloantibody titer is ≥16, said critical level of anti-Rhc maternal alloantibody titer is ≥16, said critical level of anti-RhE maternal alloantibody titer is ≥16, and said critical level of anti-RhC maternal alloantibody titer is ≥16.

10. The method of claim 1, wherein the fetal subject is a fetus with or at risk for severe HDFN.

11. The method of claim 1, wherein the anti-FcRn antibody is administered at about 15 mg/kg to about 60 mg/kg based on the weight of the pregnant woman.

12. (canceled)

13. The method of claim 11, wherein the anti-FcRn antibody is administered at about 45 mg/kg based on the weight of the pregnant woman.

14. The method of claim 11, wherein the composition is administered weekly for at least 20 weeks, and first administration is based on the weight of the pregnant woman on the day of the first administration, and each subsequent administration is based on the weight of the pregnant woman assessed a week before or two weeks before the subsequent administration.

15.-17. (canceled)

18. The method of claim 1, wherein the composition is administered:
(i) weekly for a minimum of 20 weeks and a maximum of 23 weeks;
(ii) weekly from gestational week 13, week 14, or week 15;
(iii) weekly until gestation week 35;
(iv) weekly until delivery; or
(v) weekly for at least 20 weeks, and the pregnant woman has a FcRn receptor occupancy of at least 90% after first administration of the composition, and the pregnant woman maintains a FcRn receptor occupancy of at least 90% until after last administration of the composition.

19-23. (canceled)

24. The method of claim 1, wherein the pregnant woman has obstetrical history comprising a previous pregnancy with a previous fetus, and the fetal subject shows a reduction in severity of HDFN compared with the previous fetus, or a previous pregnancy with a previous fetus, and the fetal subject shows an improvement in MCA-PSV MoM score compared with the previous fetus.

25. (canceled)

26. (canceled)

27. The method of claim 1, wherein the method treats or ameliorates one or more symptoms in the fetal subject selected from: mild anemia, severe anemia, severe anemia with enlargement of the liver and spleen, hyperbilirubinemia, jaundice, hydrops fetalis, severe hyperbilirubinemia, kernicterus, or combinations thereof.

28.-29. (canceled)

30. The method of claim 1, wherein the fetal subject is liveborn and the pregnant woman received no IUT before gestational week 24.

31. The method of claim 1, wherein the fetal subject is liveborn at or after gestational week 32 and the pregnant woman received no IUT during pregnancy with the fetal subject.

32.-44. (canceled)

45. The method of claim 1, wherein the composition is administered intravenously.

46.-50. (canceled)

51. The method of claim 1, wherein the anti-FcRn antibody comprises a light chain variable region comprising an amino acid sequence of SEQ ID NO: 9.

52. The method of claim 1, wherein the anti-FcRn antibody comprises a heavy chain variable region comprising an amino acid sequence of SEQ ID NO: 10.

53. The method of claim 1, wherein the anti-FcRn antibody comprises:
a light chain comprising the sequence of SEQ ID NO: 1; and
a heavy chain comprising the sequence of SEQ ID NO: 2.

54. The method of claim 1, wherein the anti-FcRn antibody is nipocalimab.

55.-78. (canceled)
